(19) United States
(12) Patent Application Publication
(10) Pub. No.: US 2024/0352541 A1
(43) Pub. Date: Oct. 24, 2024

(54) METHODS FOR TARGET DNA DETECTION USING CARBOXYLIC-FUNCTIONALIZED, CARBOHYDRATE-CAPPED METAL NANOPARTICLES

(71) Applicant: BOARD OF TRUSTEES OF MICHIGAN STATE UNIVERSITY, East Lansing, MI (US)

(72) Inventor: Evangelyn C. Alocilja, East Lansing, MI (US)

(21) Appl. No.: 18/643,382

(22) Filed: Apr. 23, 2024

Related U.S. Application Data
(60) Provisional application No. 63/461,436, filed on Apr. 24, 2023.

(51) Int. Cl. C12Q 1/689; B01L 3/00; C12Q 1/6806; C12Q 1/6834

(52) U.S. Cl. CPC C12Q 1/689; B01L 3/502761; C12Q 1/6834; B01L 2200/0668; B01L 2200/16; B01L 2300/0654; B01L 2300/087; C12Q 1/6806

(57) ABSTRACT

The disclosure relates to methods, apparatus, and compositions for analyzing a sample for the presence or absence of a target DNA analyte. A sample mixture is formed, which includes a sample to be analyzed and a nanoparticle-probe adduct. The nanoparticle-probe adduct is a non-covalently-bound adduct between a functionalized carbohydrate-capped metal nanoparticle and a functionalized oligonucleotide probe specific to the target DNA analyte. Upon thermal treatment of the sample mixture, a nanoparticle-probe-DNA complex forms when the target DNA analyte is present in the sample. Upon addition of a destabilizing agent (such as an acid) to the thermally treated sample mixture, remaining non-complexed nanoparticle-probe adduct will aggregate or otherwise becomes destabilized, creating a rapidly detectable color change that can be detected and correlated to the presence or absence of the target DNA in the original sample. Related apparatus, kits, and compositions for performing the methods are also disclosed.

### CROSS REFERENCE TO RELATED APPLICATION

Priority is claimed to U.S. Provisional Application No. 63/461,436, filed Apr. 24, 2023, which is incorporated herein by reference in its entirety.

### STATEMENT OF GOVERNMENT INTEREST

None.

### BACKGROUND OF THE DISCLOSURE

### Field of the Disclosure

The disclosure relates to methods, apparatus, and compositions for analyzing a sample for the presence or absence of a target DNA analyte in a sample mixture containing the sample and a metal nanoparticle-oligonucleotide probe adduct specific to the target DNA of interest. Upon incubation of the sample mixture, a nanoparticle-probe-DNA complex forms when the target DNA analyte is present in the sample. The complex remains stable upon addition of an acid to the incubated mixture, while non-complexed adduct will aggregate to create a detectable color change.

### Background

Each year, foodborne illnesses are responsible for hundreds of millions of cases around the world, with many ending in hospitalization or death. Some of the most implicated bacterial species include Salmonella spp., Campylobacter, and Shiga toxin-producing Escherichia coli (STEC). In the United States alone, STEC infections were responsible for thousands of illnesses and 660 hospitalizations in 2019. STEC outbreaks can occur in a variety of food matrices, including meat products, raw flour, and leafy green vegetables.

However, many obstacles still exist for rapid and accessible detection of foodborne pathogens. Traditional enumerative techniques typically require days of sample culturing before detection. Rapid methods have been developed to protect consumers more effectively. Widely implemented polymerase chain reaction (PCR) techniques, for instance, have reduced detection time to only hours. Despite their advantages, PCR assays also require costly reagents, advanced laboratory equipment, and trained personnel that reduce accessibility in many low-income and middle-income countries. Immunological assays such as ELISA, which rely on antibody-antigen reactions, also require highly qualified personnel as well as antibodies that increase cost and storage needs.

Colorimetric gold nanoparticle (GNP) biosensors are one potential solution. Nanoparticle properties, including a high surface area to volume ratio, make them widely applicable in analyte capture and sensing applications. GNPs are easily modified with biomolecules and are chemically stable. They also feature unique optical properties. The coherent oscillation of free electrons in colloidal GNP solutions produces a strong SPR (Surface Plasmon Resonance) band. As this SPR band is distance dependent, aggregation of the nanoparticles leads to a visible color change. Small and dispersed gold nanoparticles will feature a peak absorbance around 520 nm and appear red in color, while the aggregation of particles will lead to higher peak wavelength absorbance (approximately 600 or higher) and a visible color change to blue or purple. As a result of these properties, GNPs are utilized in a variety of biosensing techniques, including piezoelectric biosensors, fluorescence sensing, optical biosensors, and electrochemical techniques. The visible GNP color change allows for detection without expensive analytical equipment through colorimetric biosensors.

GNP colorimetric biosensors rely on the visible color change of a solution due to the aggregation of GNPs. Methods with non-target aggregation typically use one probe sequence attached to GNPs that will bind to target DNA. After DNA hybridization has occurred, a salt is added to the solution. GNPs are typically coated with adsorbed negative ions such as citrate or dextrin, whose electrostatic repulsion prevents particle aggregation; however, introduction of a salt to the colloid GNPs is known to disrupt these forces and induce particle aggregation. While GNP-probe complexes bound to target DNA are protected from aggregation and remain red in color, samples without target DNA will aggregate and turn purple or blue.

Colorimetric GNP biosensors have been implemented for detection of a variety of targets, including enzymes, ions, and viral DNA. For food pathogen detection, select biosensors have been successfully developed, for example: a biosensor able to detect 9 pg/μL of Klebsiella pneumoniae in under an hour; a biosensor able to detect 9.4 ng/μL of uropathogenic E. coli strains from pure culture in 30 min; a biosensor able to detect 10 CFU/g of Salmonella spp. in blueberry and chicken samples after pre-treatment with IMS (immunomagnetic separation) and a 6 h sample incubation. However, colorimetric GNP biosensors still face challenges for foodborne pathogen detection. For instance, limited studies have been conducted on the application of these biosensors for pathogen detection directly from foods, and pre-treatment culturing steps of at least 6 h may still be required. In addition, existing methods require days for GNP functionalization with the oligonucleotide probe, increasing the required labor for this assay.

U.S. Pub. No. 2019/0346433 is directed to metal nanoparticle compositions and their methods of formation and use, in particular gold nanoparticles (AuNP) and gold-coated magnetic nanoparticles. Compositions include aqueous suspensions of metal nanoparticles that are stabilized with one or more carbohydrate capping agents and/or that are functionalized with one or more binding pair members for capture/detection of a target analyte.

U.S. Pub. No. 2020/0132693 is directed to a method for specific detection of a target analyte using probe DNA specific to the target analyte and non-functionalized, carbohydrate-capped metal nanoparticles such as non-functionalized, dextrin-capped gold nanoparticles. A sample mixture including a target DNA analyte and a probe DNA specific thereto is incubated to form a probe DNA-target DNA complex. The non-functionalized, carbohydrate-capped metal nanoparticles and an ionic species such as sodium chloride or other salt are added to the probe DNA-target DNA complex, and the mixture is incubated. Addition of the ionic species creates a detectable distinction, such as color of the resultant mixture.

U.S. Pub. No. 2021/0164970 is directed to functionalized magnetic particle compositions and related methods to extract biological target analytes such as bacteria from samples such as clinical, industrial, or environmental samples. The functionalized magnetic particles can be synthesized in a one-pot method and include a biomimetic binding pair member which permits non-specific binding to one or more biological target analytes, such as when using the functionalized magnetic particles to extract pathogens or other analytes from a sample matrix.

### SUMMARY

In one aspect, the disclosure relates to a method for detection of a target DNA analyte, the method comprising: combining (i) a sample containing or suspected of containing a target DNA analyte with (ii) an aminated (or amine-functional) oligonucleotide probe (e.g., probe DNA) that is complementary to the target DNA analyte and (iii) a carboxylic-functionalized carbohydrate-capped metal nanoparticle (e.g., MUA or MUDA-functionalized, dextrin-capped AuNP), thereby forming a sample mixture and a nanoparticle-probe adduct in the sample mixture; incubating the sample mixture (e.g., denaturing and then annealing) under conditions sufficient to bind the oligonucleotide probe with any target DNA analyte (e.g., via hybridization) present in the sample mixture, thereby forming an incubated solution comprising (i) a nanoparticle-probe-DNA complex when the target DNA analyte is present in the sample, and (ii) the nanoparticle-probe adduct when the target DNA analyte is not present in the sample; and combining the incubated solution with an acid (e.g., adding an acid thereto) in an amount sufficient to (i) destabilize (e.g., aggregate, agglomerate, precipitate, etc.) any nanoparticle-probe adduct present in the incubated solution, and (ii) stabilize (or maintain stability for a sufficient period for measurement before eventual destabilization) any nanoparticle-probe-DNA complex present in the incubated solution.

The nanoparticle-probe adduct (or metal nanoparticle-oligonucleotide probe adduct) generally forms instantaneously and in situ when the aminated oligonucleotide probe (such as aminated probe DNA) and the carboxylic-functionalized carbohydrate-capped metal nanoparticle are combined in the sample mixture or other aqueous liquid medium, due to non-covalent interactions. The non-covalent interactions generally include hydrogen bonding between (i) the amino group (e.g., NH₂ for a primary amino group) of the oligonucleotide probe and one or both of (ii) the hydroxyl group (OH) and/or carbonyl group (C═O) of the carboxylic-functionalized carbohydrate-capped metal nanoparticle. There is typically no amide or other covalent link formed between the amino and carboxylic groups (or between the probe and metal nanoparticle more generally). In particular, a covalent link would be too strong to permit subsequent metal nanoparticle destabilization via acid treatment when there is no bound target DNA (i.e., leaving the probe DNA still attached to the metal nanoparticle and stabilizing it in solution vs. permitting aggregation). In contrast, the non-covalent interactions (e.g., in the nanoparticle-probe adduct) can be rapidly disrupted by acid treatment, thus permitting a rapidly detectable destabilization/aggregation result when there is no bound target DNA. To suspend the target DNA and form the nanoparticle-probe adduct, the sample mixture is generally formed/maintained at ambient conditions (e.g., about 25° C. or about 20° C.-30° C.) in an (aqueous) liquid medium such as phosphate buffer saline (PBS) at about pH 7.4, nuclease-free water, deionized water, or buffer used in a DNA extraction kit.

The incubated solution can generally include one or more of the nanoparticle-probe-DNA complex, the nanoparticle-probe adduct, and non-target DNA from the sample matrix. When there is no target DNA analyte present in the sample, the resulting incubated solution contains only the nanoparticle-probe adduct and non-target DNA. When there is an excess of target DNA analyte present in the sample, essentially all of the original nanoparticle-probe adduct initially formed in situ will be converted to the nanoparticle-probe-DNA complex (e.g., potentially with excess unbound target DNA remaining) and there is essentially no nanoparticle-probe adduct remaining in the incubated sample. When there is some target DNA analyte present in the sample, but at an intermediate amount, the incubated solution can contain a combination of the nanoparticle-probe-DNA complex (e.g., with essentially no unbound target DNA remaining) and the nanoparticle-probe adduct. The foregoing range of options also applies to the relative degree of stabilization, destabilization, and/or color change after addition of the acid to the incubated solution.

Various refinements, embodiments, etc. for the disclosed methods for detecting target analytes (e.g., target DNA, target RNA) are possible.

In a refinement, the method comprises detecting a relative degree of metal nanoparticle stabilization after combining the incubated solution with the acid (e.g., detecting absolute or relative amounts of destabilized nanoparticle-probe adduct and stabilized nanoparticle-probe-DNA complex). In a further refinement, detecting a relative degree of nanoparticle stabilization can comprise detecting a color state of the incubated solution after combination with the acid. A stabilized nanoparticle-probe-DNA complex generally retains (for a sufficiently long period to facilitate measurement) the original color of the carboxylic-functionalized carbohydrate-capped metal nanoparticle (e.g., red for AuNPs in general or MUA or MUDA-functionalized, dextrin capped AuNPs) as originally formed or as added to the initial sample mixture. A destabilized nanoparticle-probe adduct generally changes from the original color (e.g., red) of the carboxylic-functionalized carbohydrate-capped metal nanoparticle (e.g., to blue or purple for destabilized AuNPs). The destabilization of the nanoparticle-probe adduct occurs more rapidly than an eventual destabilization of the nanoparticle-probe-DNA complex after an extended period. A relative degree of color change or color intensity (e.g., measured spectrophotometrically at one or more characteristic wavelengths for the initial and final colors) can be calibrated to or otherwise reflect a concentration or relative amount of the target DNA in the sample. Color change and/or color intensity also can be measured optically with a camera and image analysis software, such as on a smart phone or other computing device with an installed corresponding software application.

In a refinement, the target DNA analyte comprises double-stranded genomic DNA (dsDNAg) characteristic of a target analyte organism (e.g., a pathogenic target organism). In a further refinement, the target analyte organism can be selected from the group consisting of a virus, a bacterium, a mould, a fungus, and a parasite.

In a refinement, the sample comprises a cell containing DNA (e.g., a cell from an unknown organism to identify the organism; a cell from a known patient, animal, plant, or organism to determine the presence or absence of trait in the cell, such as in a cancer or other diagnostic).

In a refinement, the sample is selected from the group consisting of biological samples, environmental samples (e.g., water, soil), industrial samples, and food samples (solid or liquid foods in raw or processed form).

In a refinement, the sample comprises a biological sample. The biological sample can be selected from the group consisting of saliva, sputum, whole blood, serum, plasma, urine, tears, skin, cerebrospinal fluid (CSF), milk, cell cultures, viruses, bacteria, moulds, and fungi (e.g., spores therefrom), parasites, tumors, plants, and food products.

In a refinement, the method comprises: extracting a raw sample with a glycan-coated magnetic nanoparticle (e.g., chitosan-coated MNP), thereby forming a sample concentrate; and combining the sample concentrate, a portion thereof, or a fraction thereof (e.g., organism or genomic DNA extracted from the sample concentrate before analysis) with the aminated oligonucleotide probe and the carboxylic-functionalized carbohydrate-capped metal nanoparticle to form the sample mixture. The sample concentrate can be rinsed or washed, for example with water or a suitable buffer (e.g., PBS), thereby retaining material potentially containing target organism or DNA in the concentrate, but eliminating the bulk of other materials from the original raw sample that could interfere with or otherwise limit the sensitivity or effectiveness of the subsequent method for detecting target DNA. Sample extraction and concentration with the glycan-coated magnetic nanoparticle can provide a universal sample preparation approach for subsequent target DNA detection, regardless the specific form of the original raw sample, because it provides a sample concentrate free or substantially free from potentially interfering materials from the original raw sample matrix. In some embodiments, the sample concentrate can be resuspended in or otherwise combined with additional liquid medium (e.g., water or suitable buffer) to increase sample volume before target DNA detection. In some embodiments, the sample concentrate or its resuspended form before DNA extraction can be incubated or cultured (e.g., for 1-8 hr or 2-6 hr) with a suitable nutrient medium (e.g., tryptic soy broth (TSB)) to increase bacterial population in the sample prior to target DNA extraction and detection, thereby increasing the amount of potential target DNA in the sample to improve the level of detection relative to the original sample. In other embodiments, the concentration effect of the extraction step and the high sensitivity of the target DNA detection method together permit the sample concentrate or its resuspended form to be analyzed directly for target DNA detection (i.e., without culturing or other amplifying step). Further details and options regarding the glycan-coated magnetic nanoparticle (or functionalized magnetic particle composition more generally) and extraction process can be found in US 2021/0164970, incorporated herein by reference.

In a refinement, the oligonucleotide probe is aminated at a 5′-end of the oligonucleotide probe. More generally, the amine functionality can be at either the 3′-end or the 5′-end of the probe. In either case, the amine functionality is suitably a primary amino group (—NH₂), but it is possible to also include secondary or tertiary amino groups. The amine functional group is generally attached/covalently bound to the probe by suitable linkers as known in the art, for example having linking groups with 2-20, 3-16, 4-12, or 6-10 carbon atoms (e.g., in an alkyl or other chain linker).

In a refinement, the oligonucleotide probe comprises a single-stranded oligonucleotide probe (e.g., ssDNAp). In a further refinement, the single-stranded oligonucleotide probe can have a length of 5 to 100 nucleotide bases.

In a refinement, the oligonucleotide probe (or ssDNAp) has a length in a range of 30 to 80 nucleotide bases. More generally, the oligo probe can have a length of at least 30, 35, 40, 45, or 50 bases and/or up to 50, 55, 60, 65, 70, 75, or 80 bases. Probe lengths in these ranges provide a good balance of target DNA specificity and sensitivity. Lower probe lengths can be highly sensitive (e.g., rapidly responding to acid treatment), but can have lower specificity to the target DNA (i.e., increasing the possibility of a false positive determination). Conversely, higher probe lengths can have reduced sensitivity (e.g., slowly responding to acid treatment), even though they have higher specificity to the target DNA (i.e., decreasing the possibility of a false positive determination).

In a refinement, the sample mixture further comprises a buffer (e.g., PBS).

In a refinement, the method does not include a DNA amplification step (e.g., no polymerase chain reaction (PCR) or other amplification such as including a plurality of denaturing/annealing/elongation cycles).

In a refinement, the carboxylic-functionalized, carbohydrate-capped metal nanoparticle comprises a gold nanoparticle, a dextrin capping agent on an outer surface of the gold nanoparticle, and a thiolated carboxylic acid on an outer surface of the gold nanoparticle (e.g., 11-MUDA or other thioalkyl carboxylic acid with the thiol group adsorbed/bound to the metal nanoparticle surface and an outwardly pointing carboxylic group). A thiol functional group is particularly suitable for attachment to a gold nanoparticle. More generally, different functional groups for attachment to a particular metal or metal alloy nanoparticle are generally known in the art and can be suitably selected by the skilled artisan. There is generally a linking group between the thiol group (or other functional group for metal nanoparticle attachment) and the carboxylic group, for example having linking groups with 2-20, 3-16, 4-12, or 6-10 carbon atoms (e.g., in an alkyl or other chain linker). The carboxylic group can be in one or more of an acid form, a salt form, and an anionic (or dissociated) form. Further details and options regarding the carbohydrate-capped metal nanoparticle can be found in US 2019/0346433, incorporated herein by reference.

In a refinement, the carboxylic-functionalized, carbohydrate-capped metal nanoparticle is free from (i) biomolecules and specific binding pair members which specifically bind to the target DNA analyte (e.g., complementary DNA or other probe covalently attached thereto), and (ii) non-specific binding pair members which non-specifically bind to the target DNA analyte (e.g., chitosan or other glycan such as in chitosan- or glycan-functionalized magnetic nanoparticles for sample extraction). This reflects the structure of the metal nanoparticles before addition to the sample mixture and in situ formation of the nanoparticle-probe adduct therein. The metal nanoparticle need only include a carboxylic group, but not any functionalizing components for specific binding of the particular target DNA analyte or for non-specific binding to biological targets in general.

In a refinement, the carboxylic-functionalized, carbohydrate-capped metal nanoparticle is in the form of a carboxylic-functionalized, stabilized metal nanoparticle suspension composition comprising: water in sufficient amount to provide an aqueous medium; and a plurality of stabilized metal nanoparticles stably suspended in the aqueous medium, each stabilized metal nanoparticle comprising: (i) a metal nanoparticle core, (ii) a carbohydrate capping agent present as a layer on an outer surface of the metal nanoparticle core in an amount sufficient to stabilize the metal nanoparticle suspension, and (iii) a carboxylic functional group on an outer surface of the metal nanoparticle core (e.g., in an amount sufficient to non-covalently bind to the oligonucleotide probe, such as with an intervening linking group between the carboxylic functional group and the metal nanoparticle surface).

In a refinement, incubating the sample mixture to form the incubated solution comprises: denaturing the sample mixture under conditions sufficient to denature any target DNA analyte present in the sample mixture; and then annealing the sample mixture under conditions sufficient to hybridize any denatured target DNA analyte present in the sample mixture with the oligonucleotide probe, thereby forming the nanoparticle-probe-DNA complex when the target DNA analyte is present in the sample.

In a refinement, combining the sample with the aminated oligonucleotide probe and the carboxylic-functionalized carbohydrate-capped metal nanoparticle comprises: combining the aminated oligonucleotide probe and the carboxylic-functionalized carbohydrate-capped metal nanoparticle (e.g., in the absence of the sample) to form the nanoparticle-probe adduct; and then combining the sample with the nanoparticle-probe adduct to form the sample mixture with the nanoparticle-probe adduct therein. In an alternative refinement, combining the sample with the aminated oligonucleotide probe and the carboxylic-functionalized carbohydrate-capped metal nanoparticle can comprise: combining the aminated oligonucleotide probe and the carboxylic-functionalized carbohydrate-capped metal nanoparticle in the presence of the sample and/or with the sample prior to formation of the nanoparticle-probe adduct (e.g., separate addition of the probe and functionalized metal nanoparticles to the sample).

In a refinement, destabilization of the nanoparticle-probe adduct present in the incubated solution can comprise agglomeration and/or aggregation of nanoparticle-probe adduct present in the incubated solution. Agglomeration is generally a reversible process (e.g., with the nanoparticles being able to be subsequently re-dispersed in solution), whereas aggregation is generally an irreversible process (e.g., with the nanoparticles being unable to be subsequently re-dispersed in solution). In either case, the clumping and/or precipitation of nanoparticles resulting from agglomeration or aggregation generates an observable or otherwise detectable color change. Agglomeration and/or aggregation can result from acid addition to the incubated solution, addition of a different destabilization agent such as a salt to the incubated solution, and/or subjecting the incubated solution to a reduction in temperature.

In a refinement, the acid comprises hydrochloric acid. More generally, the acid is not particularly limited and can include any suitable inorganic or mineral acid (e.g., hydrochloric acid, nitric acid, sulfuric acid, etc.) or organic acid (e.g., acetic acid). Alternatively or additionally, the particular acid and/or amount of acid used can be selected based on a target pH value or range in the incubated solution, for example a pH value of at least 0, 1, 2, 3, or 4 and/or up to 2, 3, 4, 5, or 6, and/or based on a relative difference in destabilization timescales for the nanoparticle-probe adduct (relatively faster) vs. the nanoparticle-probe-DNA complex. (relatively slower) The acid generally serves to remove the capping agent (e.g., dextrin or other carbohydrate) and/or the carboxylic functional group (e.g., MUA, MUDA, or otherwise), which in turn destabilizes the metal nanoparticles, causing them to aggregate and create a visible or otherwise detectable color change. When the metal nanoparticles do not have any bound target DNA (i.e., metal nanoparticles in the nanoparticle-probe adduct form), the acid works rapidly to remove the capping agent and the carboxylic functional group, creating a rapidly detectable assay result (e.g., within 5-10 minutes). In contrast, when the nanoparticles do have bound target DNA (i.e., metal nanoparticles in the nanoparticle-probe-DNA complex form), the acid can eventually remove the capping agent and the carboxylic functional group, but this removal is slower (e.g., due to the presence of the target DNA), such as requiring about 30 minutes or more, after which time even nanoparticles that originally had bound target DNA will be destabilized and will aggregate. Accordingly, a colorimetric or other determination related to nanoparticle stability is performed in a time window between the two destabilization timescales.

In another aspect, the disclosure relates to a stabilized complex suspension composition comprising: water or buffer; and a nanoparticle-probe-DNA complex stably suspended in the water or buffer: wherein: the nanoparticle-probe-DNA complex comprises: (i) a carboxylic-functionalized carbohydrate-capped metal nanoparticle, (ii) a target DNA analyte, and (iii) an aminated oligonucleotide probe that is complementary to the target DNA analyte; the oligonucleotide probe is non-covalently bound to the carboxylic-functionalized carbohydrate-capped metal nanoparticle (e.g., via the amino and carboxylic groups thereof); and the oligonucleotide probe is hybridized to the target DNA

In another aspect, the disclosure relates to a kit for detection of a target DNA analyte, the kit comprising: an aminated oligonucleotide probe that is complementary to a target DNA analyte; a carboxylic-functionalized, carbohydrate-capped metal nanoparticle; and optionally a buffer; wherein: the aminated oligonucleotide probe and the carboxylic-functionalized, carbohydrate-capped metal nanoparticle are present either in admixture or as separate components; and the aminated oligonucleotide probe and the carboxylic-functionalized, carbohydrate-capped metal nanoparticle are present in relative amounts for forming a nanoparticle-probe adduct upon combination (e.g., in selected/controlled relative nanoparticle: probe amounts or ratios (w/w or v/v), such as 1:2, 1:1.5, 1:1, 1.5:1, 2:1, or subranges thereof), the nanoparticle-probe adduct being capable of binding the target DNA analyte. In some embodiments, the kit can include multiple different aminated oligonucleotide probes for addition to/formation of a sample mixture and corresponding nanoparticle-probe adduct. The different aminated oligonucleotide probes can be combined with the nanoparticles or with other probes in selected amounts or ratios as noted above.

In another aspect, the disclosure relates to an apparatus for detecting a target DNA analyte using any of the methods as generally disclosed herein, the apparatus comprising: a sample chamber adapted to receive the sample to be analyzed for the presence or absence of the target DNA analyte (e.g., a glass or other container with transparent wall(s) or wall section(s) for visual or (spectrophotometric) optical interrogation of the sample chamber); at least one reservoir adapted to contain and deliver the aminated oligonucleotide probe and the carboxylic-functionalized carbohydrate-capped metal nanoparticle (e.g., aqueous or liquid suspension(s) thereof) to the sample chamber, either in admixture (e.g., from a single reservoir already containing the nanoparticle-probe adduct) or separately (e.g., from two different reservoirs containing the individual components such that the nanoparticle-probe adduct is formed in the sample chamber); an additional reservoir adapted to contain and deliver the acid to the sample chamber; a heating element adapted to incubate the sample mixture (e.g., denature and then anneal) in the sample chamber after delivery of the sample, the aminated oligonucleotide probe, and the carboxylic-functionalized carbohydrate-capped metal nanoparticle (e.g., as an already-formed nanoparticle-probe adduct or as separate components) to the sample chamber; optionally, an optical interrogation means adapted to colorimetrically detect metal nanoparticle destabilization and/or stabilization in the sample chamber after delivery of the acid to the incubated sample mixture (e.g., a spectrophotometric detector such as including a light source of selected or controllable wavelength(s) and a light intensity detector).

In a refinement, the apparatus can include a plurality of sample chambers, each sample chamber being adapted to (i) receive a separate sample to be analyzed for the presence or absence of the target DNA analyte, (ii) receive the aminated oligonucleotide probe and the carboxylic-functionalized carbohydrate-capped metal nanoparticle from the at least one reservoir, (iii) receive the acid from the additional reservoir, and (iv) be heated by the heating element to incubate the sample mixture therein. More generally, the apparatus can contain multiple sample chambers to analyze multiple samples for target analytes, either simultaneously or in (rapid) succession. The multiple samples can be different samples (e.g., from different or distinct sources) to be analyzed for the same target analyte, in which case only one source/reservoir for an aminated oligonucleotide probe specific to the target analyte is needed. Alternatively or additionally, the multiple samples can be aliquots or sub-samples of the same original sample (e.g., from the same source) to be analyzed for different target analytes, in which case multiple corresponding sources/reservoirs for corresponding aminated oligonucleotide probes each specific to a particular target analyte can be included.

In another aspect, the disclosure relates to a more general method for detection of a target analyte (e.g., target DNA or target RNA analyte), the method comprising: combining (i) a sample containing or suspected of containing a target DNA/RNA analyte with (ii) a functionalized oligonucleotide probe that is complementary to the target DNA/RNA analyte and that comprises a first functional group, and (iii) a functionalized carbohydrate-capped metal nanoparticle comprising a second functional group complementary to and capable of non-covalent bonding (e.g., hydrogen bonding) to the first functional group, thereby forming a sample mixture and a nanoparticle-probe adduct in the sample mixture; incubating the sample mixture (e.g., denaturing and then annealing) under conditions sufficient to bind the oligonucleotide probe with any target DNA/RNA analyte (e.g., via hybridization) present in the sample mixture, thereby forming an incubated solution comprising (i) a nanoparticle-probe-DNA/RNA complex when the target DNA/RNA analyte is present in the sample, and (ii) the nanoparticle-probe adduct when the target DNA/RNA analyte is not present in the sample; and combining the incubated solution with a destabilizing agent (e.g., acid) in an amount sufficient to (i) destabilize (e.g., aggregate, agglomerate, precipitate, etc.) any nanoparticle-probe adduct present in the incubated solution, and (ii) stabilize (or maintain stability of) any nanoparticle-probe-DNA/RNA complex present in the incubated solution. Disclosure herein specifically related to the aminated oligonucleotide probe, the target DNA analyte, the carboxylic-functionalized carbohydrate-capped metal nanoparticle, the nanoparticle-probe adduct formed therefrom, and the nanoparticle-probe-DNA complex formed therefrom can more generally apply to the functionalized oligonucleotide probe, the target DNA/RNA analyte, the functionalized carbohydrate-capped metal nanoparticle, the nanoparticle-probe adduct formed therefrom, and the nanoparticle-probe-DNA/RNA complex formed therefrom, respectively. In embodiments when the target analyte is a target RNA analyte, a sample as originally obtained (e.g., extracted from a sample matrix) can be analyzed directly for the target RNA analyte without transcription (e.g., without transcription to cDNA for subsequent detection as a target DNA analyte). In some alternative embodiments, the functionalized carbohydrate-capped metal nanoparticle can be replaced with a functionalized capped metal nanoparticle using a capping agent other than a carbohydrate capping agent, for example a citrate capping agent, such as in a carboxylic-functionalized citrate-capped gold or other metal nanoparticle. In another embodiment, the method and/or its various compositions can be free from functionalized capped metal nanoparticles using a capping agent other than a carbohydrate capping agent (e.g., free from (functionalized) citrate-capped metal nanoparticles).

In a refinement of the more general method, the first functional group is an amino group; and the second functional group is a carboxylic group. This is representative of embodiments with aminated probe DNA and carboxylic-functionalized carbohydrate-capped metal nanoparticles.

In a refinement of the more general method, the first functional group is a carboxylic group; and the second functional group is an amino group. This is representative of an alternative embodiment switching the amino and carboxylic functional groups, in particular with a carboxylic-functionalized probe (e.g., oligonucleotide probe with a terminal carboxylic group) and amino-functionalized carbohydrate-capped metal nanoparticle (e.g., gold nanoparticle functionalized with a thiolamine including a hydrocarbon linking group between the thiol group and amino group).

In a refinement of the more general method, the first functional group is either a N-hydroxysuccinimide (NHS) ester or a carboxylic group; and the second functional group is the other of the N-hydroxysuccinimide (NHS) ester and the carboxylic group (e.g., NHS ester/carboxylic groups or carboxylic/NHS ester groups as first/second groups).

In a refinement of the more general method, the first functional group is either biotin or (strept) avidin; and the second functional group is the other of biotin and (strept) avidin (e.g., biotin/(strept) avidin groups or (strept) avidin/biotin groups as first/second groups).

In a refinement of the more general method, the destabilizing agent comprises an acid (e.g., HCl or otherwise). More generally, the destabilizing agent can include a pH-adjusting agent (e.g., acid, base, pH buffer etc. to achieve a target/selected pH in the incubated solution), and/or an ionic species such as a salt (e.g., NaCl or other alkali metal (halide) salt).

In an alternative refinement of the more general method, combining the incubated solution with a destabilizing agent can more generally comprise destabilizing any nanoparticle-probe adduct present in the incubated solution, while stabilizing (or maintaining the stability of) any nanoparticle-probe-DNA/RNA complex present in the incubated solution. Destabilization, for example including agglomeration and/or aggregation, can result from acid addition to the incubated solution, addition of a different destabilization agent such as a salt to the incubated solution, and/or subjecting the incubated solution to a reduction in temperature (e.g., with or without acid or other destabilization agent addition). In some embodiments, destabilization of the nanoparticle-probe adduct can be effected by rapid cooling, such as by exposing the incubated sample to a low temperature substantially below ambient temperature (e.g., 20-30° C. or about 25° C.), for example up to −15, −10, −5, 0, 5, or 10° C. and/or at least −30, −20, −10, −5, 0, or 5° C. The incubated sample (e.g., aqueous medium thereof) can, but need not, freeze during exposure to a low temperature sufficient to cause destabilization of the nanoparticle-probe adduct. In cases where the incubated sample does freeze, destabilization and its corresponding color change are still observed, for example when the incubated sample thaws and returns to its liquid state. Similarly, the nanoparticle-probe-DNA/RNA complex remains stabilized both before and after being subjected to a reduced temperature, whether entering a frozen state or simply a reduced-temperature liquid state.

While the disclosed articles, apparatus, methods, and compositions are susceptible of embodiments in various forms, specific embodiments of the disclosure are illustrated (and will hereafter be described) with the understanding that the disclosure is intended to be illustrative, and is not intended to limit the claims to the specific embodiments described and illustrated herein.

### BRIEF DESCRIPTION OF THE DRAWINGS

For a more complete understanding of the disclosure, reference should be made to the following detailed description and accompanying drawings wherein:

FIG. 1 illustrates the detection method according to the disclosure. The first and third rows labeled with “A” illustrate the method when target DNA analyte is present in the sample being analyzed. The second and fourth rows labeled with “B” illustrate the method when non-target DNA analyte (or no target DNA analyte) is present in the sample being analyzed.

FIG. 2 includes graphs illustrating representative spectrophotometric results for a stabilized suspension of gold nanoparticles (panel A) and a de-stabilized or aggregated suspension of gold nanoparticles (panel B).

FIG. 3 illustrates an apparatus for detecting a target DNA analyte according to the disclosure.

FIG. 4 illustrates a kit for detection of a target DNA analyte according to the disclosure.

FIG. 5 is a graph illustrating the paired mean difference between target (E. coli O157) and non-target (Listeria spp.) peak absorbance wavelength at concentrations ranging from 20 ng/μL to 1.25 ng/μL (nine replicates; error bars represent 95% confidence intervals).

FIG. 6 is a graph showing the plasmonic biosensor results of E. coli samples in a bar graph plotting the average absorbance ratio at 625 and 520 nm (A_{625/520}) of the target (T) and non-target (NT) samples with colorimetric results in tubes.

### DETAILED DESCRIPTION

The disclosure relates to methods, apparatus, and compositions for analyzing a sample for the presence or absence of a target DNA analyte. A sample mixture is formed, which includes a sample to be analyzed and a nanoparticle-probe adduct. The nanoparticle-probe adduct is a non-covalently-bound adduct between a functionalized carbohydrate-capped metal nanoparticle and a functionalized oligonucleotide probe specific to the target DNA analyte. Upon thermal treatment of the sample mixture, a nanoparticle-probe-DNA complex forms when the target DNA analyte is present in the sample. Upon addition of a destabilizing agent (such as an acid) to the thermally treated sample mixture, remaining non-complexed nanoparticle-probe adduct will aggregate or otherwise becomes destabilized, creating a rapidly detectable color change that can be detected and correlated to the presence or absence of the target DNA in the original sample. Related apparatus, kits, and compositions for performing the methods are also disclosed.

### Terms

The term “ssDNA” includes a single free strand of polymerized deoxyribonucleic acids consisting of repeated polymer bases of adenine (A), cytosine (C), guanine (G), and/or thymine (T), where each strand has directionality and runs from five prime (5′) to three prime (3′)

The term “dsDNA” includes a complex of two ssDNA strands that are hybridized to each other in a complimentary fashion (adenine: thymine and cytosine: guanine), the two strands run anti-parallel to each other and form a helical structure, such that at any given end a 5′-end from one strand and a 3′-end from another strand are present.

The term “oligonucleotide” or “strand” includes a DNA molecule having from 2, 4, 6, 8, or 10 bases to 20, 50, 100, 200, 500, or 1000 bases in length and being single stranded.

The term “sequence” includes the specific nucleotide base configuration in a linear 5-prime to 3-prime order.

The term “hybridization” includes the pairing of two oligonucleotides together, where non-covalent bonding occurs between adenine and thymine or cytosine and guanine pairs, and the hybridized oligonucleotides are in opposing orientations during hybridization. Hybridization further can refer to the process of establishing a non-covalent, sequence-specific interaction between two or more complementary strands of nucleic acids into a single hybrid (e.g., via pairwise interactions between nucleic bases A=T and G=C), which can be referred to a complex (or a duplex in the case of two strands). Hybridization can be performed by incubating a sample containing complementary oligonucleotide strands at generally mild temperatures, such about room temperature (e.g., at least 10, 15, or 20° C. and/or up to 20, 25, or 30° C., such as at about 20° C. or 25° C.). A sufficient time for hybridization is not particularly limited and generally depends on the kinetics of the binding interaction for a particular pair of complementary strands (e.g., at least 0.5, 1, 2, 5, or 10 minutes and/or up to 2, 5, 10, 20, 30, 60, 90, or 120 minutes).

The term “denaturation” (or “melting”) includes a (reversible) process in which double-stranded DNA (dsDNA) unwinds and separates into single-stranded strands (complementary ssDNA). Denaturation can be performed at relatively high temperatures, for example by heating to at least 70, 80, or 90° C. and/or up to 80, 90, 95, 98, or 100° C. (e.g., at about 95° C.). A sufficient time for denaturation is not particularly limited and generally depends on the kinetics of the binding interaction for a particular pair of complementary strands (e.g., at least 0.5, 1, 2, 5, or 10 minutes and/or up to 2, 5, 10, 20, 30, 60, 90, or 120 minutes).

The term “annealing” includes the re-formation of double-stranded DNA from denatured DNA, for example between an ssDNA from an original ssDNA sample and a probe ssDNA sequence. Annealing can be performed by heating at moderate temperatures, for example by heating to at least 40, 50, or 60° C. and/or up to 50, 55, 60, 65, or 70° C. (e.g., at about 50° C., 55° C., or 60° C.). A sufficient time for annealing is not particularly limited and generally depends on the kinetics of the binding interaction for a particular pair of complementary strands (e.g., at least 0.5, 1, 2, 5, or 10 minutes and/or up to 2, 5, 10, 20, 30, 60, 90, or 120 minutes).

The term “probe” or “oligonucleotide probe” includes an oligonucleotide, generally an ssDNA probe oligonucleotide (or “ssDNAp”) having a sequence selected to hybridize to the target nucleic acid or DNA (e.g., genomic DNA), which can be characteristic to a specific organism, such as a virus, bacterium, mould, fungus, plant, prokaryote, eukaryote, or other (biological) pathogen of interest. As described in more detail, the oligonucleotide probe generally includes a functional group such as amino group or a carboxylic group for non-covalent, rapid binding and de-binding with a complementary

The term “genomic DNA” (or “gDNA”) includes chromosomal DNA, for example including the DNA carried in an organism for normal life-giving functions, where the set of DNA is specific and unique to each organism (e.g., virus, bacterium, mould, fungus, plant, prokaryote, eukaryote, or other (biological) pathogen of interest).

The term “AuNP” includes gold nanoparticles, which can be a solid gold sphere with a diameter of 5 to 50 nanometers.

The term “complementary” with respect to an oligonucleotide or DNA sequence includes a second sequence of DNA bases that mirrors a first sequence, with the second sequence having the following substitutions adenine (A) in place of thymine (T), cytosine (C) in place of guanine (G), thymine (T) in place of adenine (A) and guanine (G) in place of cytosine (C) in an anti-parallel direction relative to the first sequence.

### Detection Method

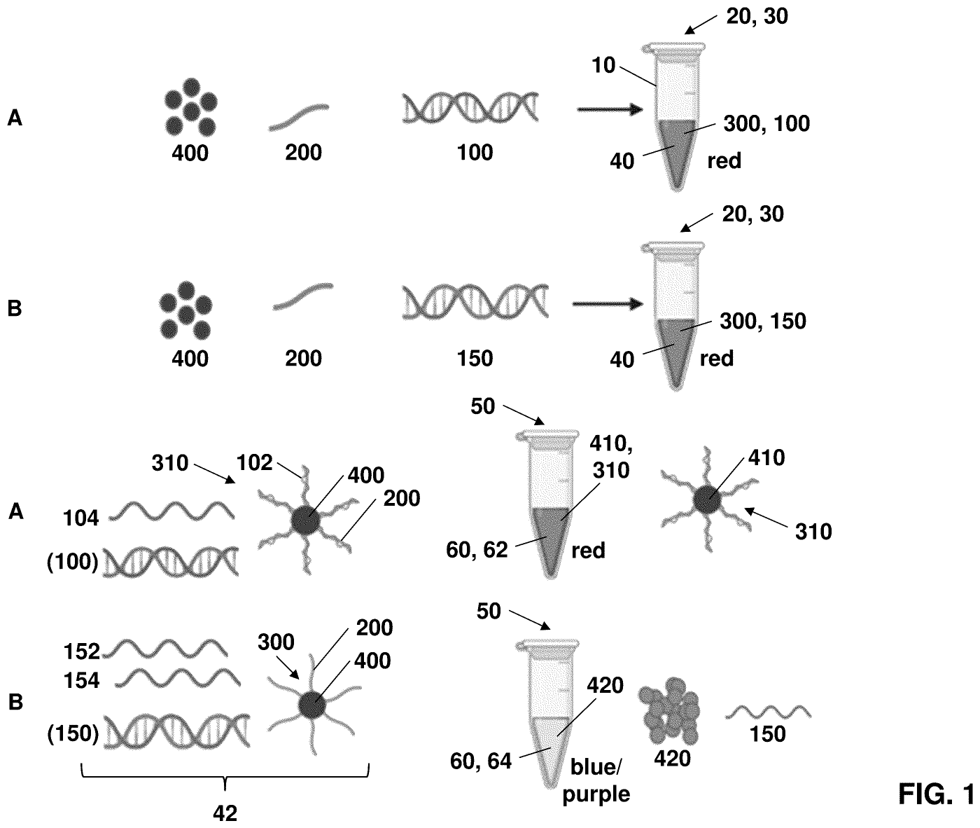

The disclosure relates to a method for detecting a target analyte as generally illustrated in FIG. 1. The method generally includes combining a sample 20 containing or suspected of containing a target DNA analyte 100 (e.g., double-stranded DNA (dsDNA)) with a functionalized oligonucleotide probe 200 (e.g., aminated or other functionalized probe DNA) that is complementary to the target DNA analyte 100 and with functionalized carbohydrate-capped metal nanoparticles 400 (e.g., carboxylic or other functionalized, dextrin-capped gold nanoparticles). The probe 200 and the metal nanoparticles 400 include complementary functional groups capable of (reversible) non-covalent bonding. The target DNA analyte 100, the probe 200, and the metal nanoparticles 400 can be combined, for example, in a sample or assay vessel 10 to form a corresponding sample mixture 40 including the target DNA analyte 100, the probe 200, and the metal nanoparticles 400. The sample 20 can contain other non-target components, for example a non-target DNA 150 (e.g., double or single stranded) as well as other potential non-DNA components from the original sample.

The sample 20 generally can include an aliquot of any matter containing, or suspected of containing, the target analyte/nucleic acid (e.g., target or genomic DNA 100) of interest. For example, samples can include biological samples, such as samples from taken from animals (e.g., saliva, whole blood, serum, plasma, urine, tears, milk, skin, cerebrospinal fluid (CSF), and the like), cell cultures, tumors, plants, virus, bacterium, mould, fungus (e.g., spores therefrom); environmental samples (e.g., water); industrial samples; and food samples or food products (e.g., solid or liquid foods in raw or processed form, such as milk). Samples may be required to be prepared prior to analysis according to the disclosed methods. For example, samples may require extraction, concentration, dilution, filtration, centrifugation, and/or stabilization prior to analysis. For the purposes herein, “sample” can refer to either a raw sample as originally collected or a sample resulting from one or more preparation techniques applied to the raw sample. For example, a raw sample can be first extracted with a glycan-coated magnetic nanoparticle as described herein to form a sample concentrate. The probe 200 and metal nanoparticles 400 can then be combined with the sample concentrate, a portion thereof, or a fraction thereof (e.g., genomic DNA extracted from the sample concentrate before analysis) to form the sample mixture 40. In various embodiments, a sample 20 to be tested by contact with probe 200 and metal nanoparticles 400 can be a liquid (e.g., aqueous) medium containing or suspected of containing the analyte, where the liquid medium can be the raw sample to be tested, or it can be a liquid medium (e.g., a PBS, biological, or other buffer) to which a solid or liquid raw or prepared sample to be tested is added.

In an embodiment, the target DNA analyte 100 includes a cell containing DNA. For example, the cell can be from an unknown organism, and the detection method described herein can be used to identify the organism. Alternatively, the cell can be from a known patient, animal, plant, or organism, and the detection method described herein can be used to determine the presence or absence of trait in the cell, such as in a cancer or other diagnostic.

In an embodiment, the target DNA analyte 100 includes double-stranded genomic DNA (dsDNAg) characteristic of a target analyte organism, such as a pathogenic target organism. For example, the target analyte organism can be a virus, a bacterium, a mould, a fungus, a parasite, or a plant. Alternatively or additionally, the target analyte organism can be a plant, human, or other animal pathogen, for example a virus, bacterium, mould, or fungus that can injure, damage, or kill a plant, human, or other animal.

In an embodiment, the functionalized oligonucleotide probe 200 is a single-stranded probe DNA (ssDNAp), where the ssDNAp is complementary to a portion of one of the strands in the target DNA analyte 100. More specifically, the probe DNA 200 (ssDNAp) can include a first oligonucleotide sequence that is complementary to and capable of hybridizing with a region of the target nucleic acid or analyte 100, for example at a first range of base positions in the target nucleic acid. The length of the probe 200 or the first oligonucleotide sequence thereof is not particularly limited, but it may be selected to have a suitable length such as from 5 to 100 nucleotide bases (e.g., at least 5, 10, 15, 20, or 30 and/or up to 10, 20, 30, 40, 60, 80, or 100 bases). In some embodiments, the probe 200 (or ssDNAp) can have a length in a range of 30 to 80 nucleotide bases. More generally, the probe can have a length of at least 30, 35, 40, 45, or 50 bases and/or up to 50, 55, 60, 65, 70, 75, or 80 bases. Probe lengths in these ranges provide a good balance of target DNA specificity and sensitivity. Lower probe lengths can be highly sensitive (e.g., rapidly responding to acid treatment), but can have lower specificity to the target DNA (i.e., increasing the possibility of a false positive determination). Conversely, higher probe lengths can have reduced sensitivity (e.g., slowly responding to acid treatment), even though they have higher specificity to the target DNA (i.e., decreasing the possibility of a false positive determination).

The probe 200 includes a functional group that is complementary to and capable of non-covalent bonding (e.g., hydrogen bonding) to a functional group on the metal nanoparticles 400. For example, the functional group for probe 200 can be an amino group (e.g., NH₂ for a primary amino group), such as when the corresponding functional group of the metal nanoparticles 400 is a carboxylic group. In such cases, the amino group can exhibit non-covalent interactions (e.g., hydrogen bonding) with one or both of the hydroxyl group (OH) and/or carbonyl group (C═O) of the carboxylic group. Other functional groups for the oligonucleotide probe 200 are possible. For example, the functional group of the oligonucleotide probe 200 can be a carboxylic group, and the functional group of the metal nanoparticles 400 can be an amino group. In another embodiment, the functional group of the oligonucleotide probe 200 can be either a N-hydroxysuccinimide (NHS) ester or a carboxylic group, and the functional group of the metal nanoparticles 400 can be the other of the N-hydroxysuccinimide (NHS) ester and the carboxylic group (e.g., NHS ester/carboxylic groups or carboxylic/NHS ester groups as complementary groups for non-covalent bonding). In another embodiment, the functional group of the oligonucleotide probe 200 can be either biotin or (strept) avidin, and the functional group of the metal nanoparticles 400 can be the other of biotin and (strept) avidin (e.g., biotin/(strept) avidin groups or (strept) avidin/biotin groups as complementary groups for non-covalent bonding).

In an embodiment, the oligonucleotide probe 200 is functionalized (e.g., aminated) at a 5′-end of the oligonucleotide probe. More generally, the amine or other functionality can be at either the 3′-end or the 5′-end of the probe. The amine functionality is suitably a primary amino group (—NH₂), but it is possible to also include secondary or tertiary amino groups. The amine or other functional group is generally attached/covalently bound to the probe by suitable linkers as known in the art, for example having linking groups with 2-20, 3-16, 4-12, or 6-10 carbon atoms (e.g., in an alkyl or other chain linker). For example, oligonucleotide probes having a selected or desired oligonucleotide sequence along with a functional group such as 5′-amino group are commercially available from Integrated DNA Technologies (Coralville, IA).

The probe 200 can be labeled (e.g., with an attached enzyme, chromogenic substrate, chromophore, radioisotope, fluorescent molecule, phosphorescent molecule, chemiluminescent molecule, metal nanoparticle, polymeric nanoparticle) or unlabeled (e.g., without any of the foregoing attached components). The probe 200 suitably is unlabeled.

In an embodiment, the sample mixture 40 can further include a (pH) buffer 30, for example a buffer solution or components thereof added to an aqueous matrix of the sample matrix 20. The buffer solution 30 can generally include any suitable physiological or biological buffer, such as phosphate-buffered saline or otherwise. The sample 20, the probe 200, the metal nanoparticles 400, and the buffer 30 (when present) can be combined or added to each other in any suitable manner or order. For example, the sample 20 can be added to the probe 200 and metal nanoparticles 400 or vice versa. Similarly, the sample 20, the probe 200, and the metal nanoparticles 400 can be added to a third component or medium, such as the buffer solution 30. In a further embodiment, the buffer 30 includes a phosphate-buffered saline (PBS) buffer or solution. The PBS buffer can include disodium hydrogen phosphate and sodium chloride at any suitable concentrations. As used and as present in the sample mixture 40 with the sample 20, probe 200, metal nanoparticles 400, and any other components added to the sample mixture 40, sodium chloride can be is present in an amount in a range from 10 mM to 400 mM, such as at least 10, 20, 40, 60, 80, or 100 mM and/or up to 100, 150, 200, 250, 300, or 400 mM. Alternatively, or additionally, disodium hydrogen phosphate suitably can be present in an amount in a range from 0.5 mM to 20 mM.

Once combined, the probe 200 and the metal nanoparticles 400 together form a nanoparticle-probe adduct 300 as illustrated in FIG. 1. The nanoparticle-probe adduct 300 (or metal nanoparticle-oligonucleotide probe adduct more specifically) generally forms instantaneously and in situ when the functionalized oligonucleotide probe 200 (such as aminated probe DNA) and the functionalized carbohydrate-capped metal nanoparticles 400 (such as carboxylic-functionalized metal nanoparticles) are combined in the sample mixture 400 or other aqueous liquid medium, due to non-covalent interactions. In the illustrative case of an aminated probe 200 and carboxylic-functionalized metal nanoparticles 400, the non-covalent interactions generally include hydrogen bonding between the amino group (e.g., NH₂ for a primary amino group) of the oligonucleotide probe and one or both of the hydroxyl group (OH) and/or carbonyl group (C═O) of the carboxylic-functionalized carbohydrate-capped metal nanoparticle. There is typically no amide or other covalent link formed between the amino and carboxylic groups (or between the probe 200 and metal nanoparticles 400 more generally with other functional groups). In particular, a covalent link would be too strong to permit subsequent metal nanoparticle destabilization via acid treatment when there is no bound target DNA (i.e., leaving the probe DNA still attached to the metal nanoparticle and stabilizing it in solution vs. permitting aggregation). In contrast, the non-covalent interactions (e.g., in the nanoparticle-probe adduct 300) can be rapidly disrupted by acid treatment, thus permitting a rapidly detectable destabilization/aggregation result when there is no bound target DNA 100. To suspend the target DNA 100 and form the nanoparticle-probe adduct 300, the sample mixture 40 is generally formed or maintained at ambient conditions (e.g., about 25° C. or about 20° C.-30° C.) in an (aqueous) liquid medium such as phosphate buffer saline (PBS) at about pH 7.4, nuclease-free water, deionized water, or buffer used in a DNA extraction kit.

Formation of the nanoparticle-probe adduct 300 is illustrated in the top two rows of FIG. 1. Row A illustrates the case when the sample 20 contains the target DNA 100. Row B illustrates the case when the sample 20 contains non-target DNA 150 (e.g., and is free or substantially free from target DNA 100). In either case, the initial sample mixture 40 includes the nanoparticle-probe adduct 300, which is stabilized and evenly dispersed throughout the sample mixture 40, thus providing a characteristic red color when the metal nanoparticles 400 of the adduct 300 are gold nanoparticles. A representative structure of the nanoparticle-probe adduct 300 is shown in the bottom row of FIG. 1 and illustrated as a central carbohydrate-capped metal nanoparticle 400 non-covalently bound to a plurality of oligonucleotide probes 200 via their complementary functional groups (e.g., carboxylic and amino groups or otherwise; not shown). Regardless of whether the initial sample mixture 40 contains the target DNA 100 or the non-target DNA 150 (top rows A and B of FIG. 1, respectively), the nanoparticle-probe adduct 300 remains stabilized in the sample mixture 40.

In an embodiment, the method can include combining the functionalized oligonucleotide probe 200 and the functionalized carbohydrate-capped metal nanoparticles 400 to form the nanoparticle-probe adduct 300, for example in the absence of the sample 20. The sample 20 can then be combined with the adduct 300 to form the sample mixture 40 with the nanoparticle-probe adduct 300 therein. In another embodiment, the functionalized oligonucleotide probe 200 and the functionalized carbohydrate-capped metal nanoparticles 400 can be combined in the presence of the sample 20 and/or with the sample 20 prior to formation of the nanoparticle-probe adduct 300 (e.g., separate addition of the probe 200 and metal nanoparticles 400 to the sample 20).

The sample mixture 40 is then incubated to form an incubated sample or solution 42. More specifically, the sample mixture 40 is incubated under conditions sufficient to bind (e.g., hybridize) the oligonucleotide probe 200 component of the nanoparticle-probe adduct 300 with any target DNA analyte 100 present in the sample mixture 40, which in turn forms a nanoparticle-probe-(target) DNA complex 310 in the incubated solution 42 when the target DNA 100 analyte is present in the sample 20. When the target DNA analyte 100 is not present in the sample 20 (or present at a low, non-detectable level), the nanoparticle-probe adduct 300 remains in the incubated solution 42. In some cases, such as when the adduct 300 is present in excess relative to the target DNA 100 analyte, the incubated solution 42 can include both the nanoparticle-probe-(target) DNA complex 310 and the nanoparticle-probe adduct 300 in some relative amounts.

The incubated solution 42 can generally include one or more of the nanoparticle-probe-DNA complex 310, the nanoparticle-probe adduct 300, and non-target DNA 150 from the sample matrix. When there is no target DNA analyte 100 present in the sample 20, the resulting incubated solution 42 contains only the nanoparticle-probe adduct 300 and non-target DNA 150 (when originally present in the sample), for example including first and second individual (or denatured) strands 152, 154 of the original non-target DNA 150. When there is an excess of target DNA analyte 100 present in the sample 20, essentially all of the original nanoparticle-probe adduct 300 initially formed in situ will be converted to the nanoparticle-probe-DNA complex 310 (e.g., potentially with excess remaining unbound target DNA 100 and/or first and second individual (or denatured) strands 102, 104 thereof), and there is essentially no nanoparticle-probe adduct 300 remaining in the incubated sample 42. When there is some target DNA analyte 100 present in the sample, but at an intermediate amount, the incubated solution 42 can contain a combination of the nanoparticle-probe-DNA complex 310 (e.g., with essentially no remaining unbound target DNA 100, but possibly some second individual (or denatured) strands 104 thereof) and the nanoparticle-probe adduct 300. The foregoing range of options also applies to the relative degree of stabilization, destabilization, and/or color change after addition of the acid to the incubated solution 42.

In an embodiment, incubating the sample mixture 40 to form the incubated solution 42 can include first denaturing the sample mixture 40 under conditions sufficient to denature (e.g., at least partially unwind or de-hybridize) any (e.g., at least some or substantially all) target DNA analyte 100 present in the sample mixture 40, for example to form free first strands 102 of the target DNA analyte 100 (dsDNA target analyte) and free second strands 104 of the target DNA analyte 100 (dsDNA target analyte). Denaturation can likewise denature non-target DNA analyte 150 present in the sample mixture 40, for example to form free first strands 152 of the non-target DNA analyte 150 (dsDNA non-target analyte) and free second strands 154 of the non-target DNA analyte 150 (dsDNA non-target analyte). Denaturation can be performed at relatively high temperatures, for example by heating to at least 70, 80, or 90° C. and/or up to 80, 90, 95, 98, or 100° C. (e.g., at about 95° C.). A sufficient time for denaturation is not particularly limited, for example being at least 0.5, 1, 2, 5, or 10 minutes and/or up to 2, 5, 10, 20, 30, 60, 90, or 120 minutes. The sample mixture 40 is then annealed under conditions sufficient to hybridize any (e.g., at least some or substantially all) denatured target DNA analyte 100 present in the sample mixture 40 with the oligonucleotide probe 200 component of the nanoparticle-probe adduct 300, which in turn forms the nanoparticle-probe-(target) DNA complex 310 when the target DNA analyte 100 is present in the sample. A representative structure of the nanoparticle-probe-(target) DNA complex 310 is shown in the third row of FIG. 1 and illustrated as a central carbohydrate-capped metal nanoparticle 400 non-covalently bound to a plurality of oligonucleotide probes 200 via their complementary functional groups (e.g., carboxylic and amino groups or otherwise; not shown), with the probes 200 in turn hybridized with the first strands 102 of the target DNA analyte 100 to which they are complementary. As illustrated, the second strands 104 of the target DNA analyte 100 are not complementary with the probes 200 and the second strands 104 can remain free in the incubated solution 42. Annealing can further re-hybridize denatured/separated first and second strands 102, 104 (e.g., to re-form target DNA 100), such as where at least some first strands 102 do not hybridize with the probes 200 (e.g., when there is excess target DNA 100 relative to adduct 300). Similarly, annealing can further re-hybridize denatured/separated first and second strands 152, 154 (e.g., to re-form non-target DNA 150). Annealing can be performed by heating at moderate temperatures, for example by heating to at least 40, 50, or 60° C. and/or up to 50, 55, 60, 65, or 70° C. (e.g., at about 50° C., 55° C., or 60° C.). A sufficient time for annealing is not particularly limited and can be at least 0.5, 1, 2, 5, or 10 minutes and/or up to 2, 5, 10, 20, 30, 60, 90, or 120 minutes.

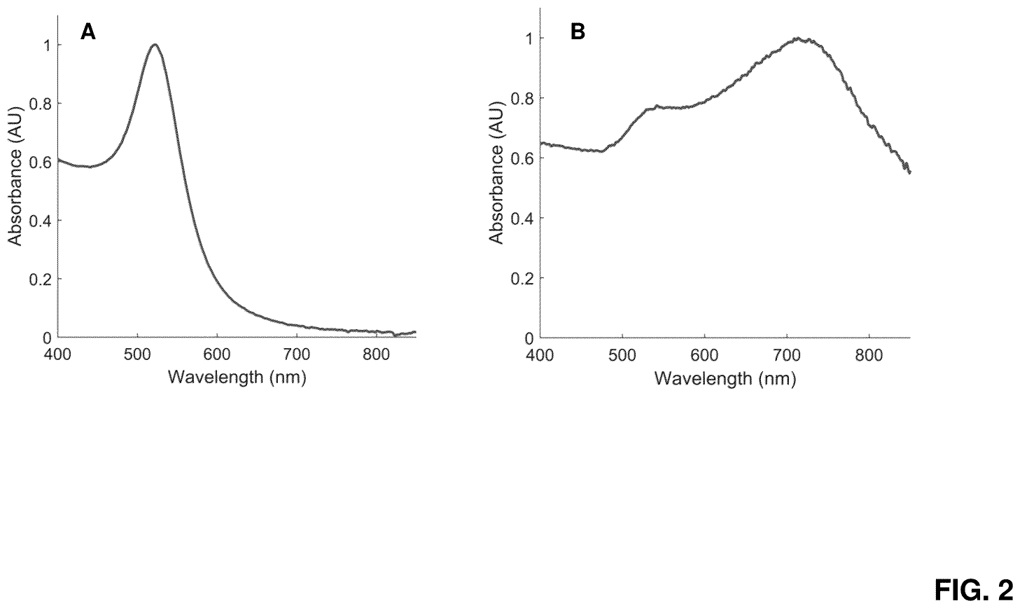

As further illustrated in FIG. 1, an acid 50 is added to the incubated solution 42 to form a solution-nanoparticle mixture 60. The acid 50 is added in an amount sufficient to destabilize any nanoparticle-probe adduct 300 present in the incubated solution 42, for example to aggregate, agglomerate, precipitate, etc. the nanoparticle-probe adduct 300 and form a destabilized solution-nanoparticle mixture 64 including destabilized metal nanoparticles 420. Similarly, the acid 50 is added in an amount sufficient to stabilize (or maintain stability for a sufficient period for measurement before eventual destabilization) any nanoparticle-probe-DNA complex 310 present in the incubated solution 42, forming a stabilized solution-nanoparticle mixture 62 including stabilized metal nanoparticles 410 (or stabilized nanoparticle-probe-target DNA complexes). As illustrated in FIG. 1 for the case of gold nanoparticles in the original metal nanoparticles 400, the stabilized solution-nanoparticle mixture 62 retains its original red color from the sample mixture 40, while the destabilized solution-nanoparticle mixture 64 changes color to a blue or purple color. FIG. 2 illustrates representative absorbance spectra results for a stabilized suspension of gold nanoparticles (panel A) and a de-stabilized or aggregated suspension of gold nanoparticles (panel B).

The acid 50 is not particularly limited and can include any suitable inorganic or mineral acid (e.g., hydrochloric acid, nitric acid, sulfuric acid, etc.) or organic acid (e.g., acetic acid). Alternatively or additionally, the particular acid and/or amount of acid used can be selected based on a target pH value or range in the incubated solution 42, for example a pH value of at least 0, 1, 2, 3, or 4 and/or up to 2, 3, 4, 5, or 6, and/or based on a relative difference in destabilization timescales for the nanoparticle-probe adduct 300 (relatively faster) vs. the nanoparticle-probe-DNA complex 310 (relatively slower) The acid generally serves to remove the capping agent (e.g., dextrin or other carbohydrate) and/or the carboxylic functional group (e.g., MUA, MUDA, or otherwise), which in turn destabilizes the metal nanoparticles, causing them to aggregate and create a visible or otherwise detectable color change. When the metal nanoparticles do not have any bound target DNA (i.e., metal nanoparticles in the nanoparticle-probe adduct 310 form), the acid works rapidly to remove the capping agent and the carboxylic functional group, creating a rapidly detectable assay result (e.g., within 5-10 minutes). In contrast, when the nanoparticles do have bound target DNA (i.e., metal nanoparticles in the nanoparticle-probe-DNA complex 310 form), the acid can eventually remove the capping agent and the carboxylic functional group, but this removal is slower (e.g., due to the presence of the target DNA 100 in the form of first strands 102 thereof hybridized with the probes 200), such as requiring about 30 minutes or more, after which time even nanoparticles that originally had bound target DNA will be destabilized and will aggregate. Accordingly, a colorimetric or other determination related to nanoparticle stability is performed in a time window between the two destabilization timescales.

In an embodiment, the method further includes detecting a relative degree of metal nanoparticle stabilization after combining the incubated solution with the acid (e.g., detecting absolute or relative amounts of destabilized nanoparticle-probe adduct and stabilized nanoparticle-probe-DNA complex). Detecting a relative degree of nanoparticle stabilization can include detecting a color state of the incubated solution after combination with the acid. A stabilized nanoparticle-probe-DNA complex generally retains (for a sufficiently long period to facilitate measurement) the original color of the carboxylic-functionalized carbohydrate-capped metal nanoparticle (e.g., red for AuNPs in general or MUA or MUDA-functionalized, dextrin capped AuNPs) as originally formed or as added to the initial sample mixture. A destabilized nanoparticle-probe adduct generally changes from the original color (e.g., red) of the carboxylic-functionalized carbohydrate-capped metal nanoparticle (e.g., to blue or purple for destabilized AuNPs). The destabilization of the nanoparticle-probe adduct occurs more rapidly than an eventual destabilization of the nanoparticle-probe-DNA complex after an extended period. A relative degree of color change or color intensity (e.g., measured spectrophotometrically at one or more characteristic wavelengths for the initial and final colors) can be calibrated to or otherwise reflect a concentration or relative amount of the target DNA in the sample. Color change and/or color intensity also can be measured optically with a camera and image analysis software, such as on a smart phone or other computing device with an installed corresponding software application.

### Functionalized, Carbohydrate-Capped Metal Nanoparticles

The disclosure relates to the use of functionalized carbohydrate-capped (e.g., stabilized) metal nanoparticle compositions, in particular gold nanoparticles (AuNPs) such as solid AuNPs or nanoparticles with a gold (shell)-nanoparticle (core) structure. The metal nanoparticles can be in the form of a metal nanoparticle core stabilized by the carbohydrate capping agent (e.g., a metal nanoparticle formed substantially entirely from gold). Alternatively, the metal nanoparticles can be in the form of a nanoparticle core (e.g., non-metallic and/or magnetic) having a metal coating in a core-shell configuration (e.g., a magnetic iron oxide-gold composite particle in a core-shell configuration), where the core-shell nanoparticle is stabilized by the carbohydrate capping agent (e.g., via interactions between the metal shell and the capping agent). In addition to the carbohydrate capping agent, the metal nanoparticles further include a functional group (e.g., carboxylic group) that is complementary to and capable of non-covalent bonding (e.g., hydrogen bonding) to a functional group (e.g., amino group) on the oligonucleotide probe. Compositions for use according to the disclosure include aqueous suspensions of metal nanoparticles that are stabilized with one or more carbohydrate capping agents. The nanoparticle suspensions are stable for extended periods (e.g., for at least several months) and can be used as desired at a later point in time, typically prior to use in an assay for the detection of a target biological analyte as described herein. The stable nanoparticle suspension can be formed by the aqueous reduction of metal precursor ions at non-acidic pH values in the presence of a carbohydrate-based capping agent such as dextrin or other oligosaccharides, followed by functionalization to attach the (pendant) functional group for non-covalent bonding and nanoparticle-probe adduct formation with the complementary functional group on the oligonucleotide probe.

Metal Nanoparticle Formation: Methods of metal nanoparticle formation according to the disclosure generally are performed in an aqueous reaction system including metal ions to be reduced in solution in the aqueous medium. The metal ions in the aqueous medium are reduced at a neutral or alkaline pH value in the presence of a carbohydrate capping agent under suitable reaction conditions to form a plurality of reduced metal nanoparticles (e.g., at a reaction temperature and reaction time sufficient to convert all or substantially all of the metal ion precursors). The reaction generally includes an initial nucleation stage to form metallic nuclei followed by a longer growth stage in which metal ions reduced on the nuclei surfaces create the final metal nanoparticles. The plurality of reduced metal nanoparticles is in the form of a stabilized suspension of metal nanoparticles in the aqueous medium, where the carbohydrate capping agent stabilizes the formed nanoparticle suspension.

The specific metal ions or oxidized metal-containing species in solution and selected as precursors to the desired metal nanoparticles are not particularly limited and are suitably chosen according to a desired end use/application of the nanoparticle suspension. In an embodiment, the metal ions include gold ions (e.g., Au(III), Au³⁺) and are selected to form gold metal nanoparticles (AuNPs). The metal ions can be free in solution or coordinated/coupled with other (ionic) species (e.g., Au³⁺, [AuCl₄]⁻, [AuCl₃OH]⁻, [AuCl₂(OH)₂]⁻, [AuCl(OH)₃]⁻, or [Au(OH)₄]⁻, where the oxidation level of gold in each case is +3). Other potential metal ions can include chromium, copper, zinc, nickel, cadmium, silver, cobalt, indium, germanium, tin, lead, arsenic, antimony, bismuth, chromium, molybdenum, manganese, iron, ruthenium, rhodium, palladium, osmium, iridium, and platinum. In some embodiments, two or more types of metal ions can be in solution in the aqueous medium to provide metal nanoparticles formed from alloys of two or more elemental metals. The concentration of metal ions in solution prior to reaction is not particularly limited, but it suitably ranges from 0.1 mM to 1000 mM (e.g., at least 0.1 mM, 1 mM, or 10 mM and/or up to 100 mM or 1000 mM).

The metal ions are suitably introduced into the aqueous medium as a dissolvable ionic compound, for example a salt or acid. A suitable source of gold ions is chloroauric acid (HAuCl₄), which can provide Au(III) in the form of [AuCl₄]. Other salts/compounds including the oxidized metal precursor such as halides (e.g., chlorides, bromides, fluorides, iodides), sulfates, sulfites, thiosulfates, nitrates, nitrites, carboxylates, sulfonates, and hydrogenated forms thereof (e.g., as in HAuCl₄) can be used as desired and depending on the particular metal ion to be introduced into the aqueous medium.

In some embodiments, the aqueous medium further includes, prior to reduction of the metal ions, a population of nanoparticles serving as cores/nucleation sites for deposition of the reduced metal ions, thus permitting the formation of metal nanoparticles having a core-shell structure including a nanoparticle core with a metallic shell. The nanoparticle core material is not particularly limited and can be non-metallic, metallic (e.g., different from the metal to be reduced as a shell), magnetic, etc. Magnetic nanoparticle cores are particularly useful to permit the resulting metal nanoparticle to function as both a magnetic sample/analyte separator and concentrator (e.g., due to the magnetic core) as well as a signal transducer (e.g., due to the electrical properties of the metal shell material such as gold).

The magnetic nanoparticles according to the disclosure are not particularly limited and generally include any nano-sized particles (e.g., about 1 nm to about 1000 nm) that can be magnetized with an external magnetic/electrical field. The magnetic nanoparticles more particularly include superparamagnetic particles, which particles can be easily magnetized with an external magnetic field (e.g., to facilitate separation or concentration of the particles from the bulk of a sample medium) and then redispersed immediately once the magnet is removed (e.g., in a new (concentrated) sample medium). Thus, the magnetic nanoparticles are generally separable from solution with a conventional magnet. Suitable magnetic nanoparticles are provided as magnetic fluids or ferrofluids, and mainly include nano-sized iron oxide particles (Fe₃O₄ (magnetite) or gamma-Fe₂O₃ (maghemite)) suspended in a carrier liquid. Such magnetic nanoparticles can be prepared by superparamagnetic iron oxide by precipitation of ferric and ferrous salts in the presence of sodium hydroxide and subsequent washing with water. A suitable source of gamma-Fe₂O₃ is Sigma-Aldrich (St. Louis, Mo.), which is available as a nano-powder having particles sized at <50 nm with a specific surface area ranging from about 50 m²/g to about 250 m²/g. Preferably, the magnetic nanoparticles have a small size distribution (e.g., ranging from about 5 nm to about 25 nm) and uniform surface properties (e.g., about 50 m²/g to about 245 m²/g).

More generally, the magnetic nanoparticles can include ferromagnetic nanoparticles (i.e., iron-containing particles providing electrical conduction or resistance). Suitable ferromagnetic nanoparticles include iron-containing magnetic metal oxides, for example those including iron either as Fe(II), Fe(III), or a mixture of Fe(II)/Fe(III). Non-limiting examples of such oxides include FeO, gamma-Fe₂O₃ (maghemite), and Fe₃O₄ (magnetite). The magnetic nanoparticles can also be a mixed metal oxide of the type M1ₓM2₃₋ₓO₄, wherein M1 represents a divalent metal ion and M2 represents a trivalent metal ion. For example, the magnetic nanoparticles may be magnetic ferrites of the formula M1 Fe₂O₄, wherein M1 represents a divalent ion selected from Mn, Co, Ni, Cu, Zn, or Ba, pure or in admixture with each other or in admixture with ferrous ions. Other metal oxides include aluminum oxide, chromium oxide, copper oxide, manganese oxide, lead oxide, tin oxide, titanium oxide, zinc oxide and zirconium oxide, and suitable metals include Fe, Cr, Ni or magnetic alloys.

Reduction of the metal ions in the aqueous medium is performed at a neutral or alkaline pH value, for example ranging from 7 to 12 (e.g., where the pH value is essentially constant throughout the reaction, or it may vary within the range during reaction). In various embodiments, the pH value of the reaction medium can be at least 7, 7.5, 8, 8.5, 9 and/or up to 8, 8.5, 9, 9.5, 10, 11, 12. The selection and control of the desired pH value can be effected by any suitable base and/or buffer system as is generally known in the art. As described below, in some embodiments, the pH value can be controlled by selection of a reducing agent. Non-acidic pH values, in particular those that are mildly basic or otherwise near to a physiological pH value, are desirable in certain embodiments to promote functionalization of the eventual metal nanoparticles with biomolecules that would be denatured or whose activity would otherwise be reduced or negated in an acidic environment.

The reaction temperature of the reduction process is not particularly limited, for example being at room temperature (e.g., 20° C. to 25° C.) or at mildly elevated temperatures relative to room temperature. In various embodiments, the temperature of the aqueous medium can range from 20° C. to 100° C. during the reduction reaction, for example being at least 20° C., 25° C., 30° C., 35° C., or 40° C. and/or up to 30° C., 35° C., 40° C., 50° C., 60° C., 70° C., 80° C., 90° C., or 100° C. in various embodiments.

Reduction of the metal ions in the aqueous medium is suitably effected by the addition of a chemical reducing agent to the aqueous medium. Suitable reducing agents are those that are effective at reducing metallic ions at the neutral/alkaline pH of the aqueous medium (e.g., they do not require an acidic pH and/or do not themselves create an acidic environment). In some embodiments, the reducing agent is a combined reducing agent for reducing the metal ions and pH-adjusting agent for maintaining the neutral or alkaline pH value of the aqueous medium. Suitable combined reducing and pH-adjusting agents include metal (e.g., alkali or alkali earth metal) carbonates or bicarbonates such as sodium carbonate (Na₂CO₃). However, other reducing agents that are operative at neutral/alkaline pH values can be used even if they do not also function as a pH-adjusting agent (e.g., in which case other non-reducing bases/buffers can be used to independently control the pH value). Examples of other suitable reducing agents include hydrides (e.g., lithium aluminum hydride (LiAlH₄), sodium borohydride (NaBH₄), diisobutylaluminum hydride (DIBAH)), dithiothreitol (DTT), sulfites/bisulfites (e.g., ammonium, metallic such as from alkali and alkali earth metals including K, Na, Li, Mg, Ba, Ca), sulfates (e.g., metallic such as from iron (II) or other soluble iron (II) salts), peroxides (e.g., those functioning as reducing agents at alkaline pH such as hydrogen peroxide (H₂O₂)), sulfides (e.g., metallic such as from alkali metals like Na), and amines (e.g., including ammonium salts thereof such as hydroxylamine (NH₂OH) or hydroxylamine hydrochloride (NH₂OH·HCl)).

The carbohydrate useful as a capping agent according to the disclosure is generally an oligo- or polysaccharide having a plurality of saccharide residues (e.g., having a general formula Cₘ(H₂O) n for unmodified carbohydrates with residues derived from monosaccharides having a general formula (CH₂O)ₙ). In some embodiments, the carbohydrate capping agent can be a carbohydrate derivative, for example having additional functional groups such as carboxylate group or nitrogen-containing groups (e.g., amino, N-acetyl). The capping agent can include linear and/or branched carbohydrates, such as those including alpha- or beta-glycosidic bonds (e.g., alpha (1,4) or alpha (1,6)glycosidic linkages as in dextrin or other starch-based capping agents). The specific carbohydrate capping agent is suitably selected so that it has at least some hydrophilic character (e.g., to promote a water-stable suspension), and it can be a water-soluble carbohydrate in some refinements. In some embodiments, the capping agent is in a substantially non-oxidized form (e.g., being (substantially) free from aldose, ketose, and/or carboxylate (acid or anion) functionalities either for a portion of or the whole capping agent molecule; based on an absence of such functionalities and/or the inability to detect (non-trace) levels of the functionalities in the capping agent), for example as added to the reaction mixture, as present during reaction, and/or as bound/conjugated to the metal nanoparticles in the reaction product. In other embodiments, other non-carbohydrate capping agents such as polyethylethene glycol (e.g., or other polyether or polyethylene oxide), various silanes, polyacrylamide, and other negatively charged polymers can be used (e.g., for use instead of or in combination with other carbohydrate capping agent such as oligosaccharide; suitably in combination with a monosaccharide, a disaccharide, or a derivative thereof as described below as an additive to the carbohydrate capping agent system). The concentration of the capping agent in solution prior to reaction is not particularly limited, but it suitably ranges from 1, 2, 5, or 10 g/L to 15, 25, 35, 50, or 100 g/L (e.g., where selection of the capping agent concentration can permit selection of an average metal nanoparticle size and/or size distribution resulting from the concentration).

The capping agent is suitably an oligosaccharide having 3 to 100 saccharide residues, for example at least 3, 5, 10, 15, 20, 25, 30, or 40 and/or up to 10, 20, 30, 40, 50, 60, 80, or 100 saccharide residues. In some embodiments, the capping agent represents a plurality of oligosaccharides or polysaccharides having a distribution of sizes/lengths (e.g., in terms of number of saccharide residues). In such cases, ranges characterizing the oligosaccharide capping agent in terms of number of saccharide residues can represent an average of the distribution (e.g., number or other average), or the ranges can represent upper and lower bounds for the distribution (e.g., within 1, 2, or 3 standards deviations from the mean; representing the 1%/99%, 5%/95%, or 10%/90% cut points of the cumulative size distribution).

In some embodiments, the carbohydrate capping agent can include one or more glucose residues (e.g., D-glucose; having a plurality of glucose residues such as where the capping agent essentially consists only of glucose residues). However, the capping agent can include other saccharide residues alone, in combination with glucose, and/or in combination with each other, for example including those from allose, altrose, mannose, gulose, iodose, galactose, talose, xylose, arabinose, fucose, and/or fructose. As noted above, the capping agent can include carbohydrate derivates, for example including saccharide residues from glucuronic acid (e.g., also including salts and esters thereof), N-acetyl-D-glucosamine (e.g., derived from chitin), and D-glucosamine (e.g., derived from chitosan).

Oligomeric carbohydrate capping agents containing the various saccharide residues can be (synthetic) oligosaccharides having a selected length/saccharide sequence, or they can be formed from naturally occurring polysaccharides. Polysaccharides can be subjected to enzymatic or other chemical forms of hydrolysis to form shorter oligosaccharides, generally with an element of random size distribution. Examples of suitable precursor polysaccharides for capping agents include starch (e.g., forming dextrin), amylose, amylopectin, cellulose, laminarin, chrysolaminarin, xylan, arabinoxylan, mannan, fucoidan, and galactomannan. In an embodiment, the capping agent is a dextrin (e.g., linear, branched, or cyclic; suitably linear and/or branched having at least 10, 20, or 30 saccharide residues), for example being formed from starch (e.g., including amylose and/or amylopectin).

In an embodiment, the aqueous medium can include a saccharide-based moiety in addition to the carbohydrate capping agent during metal ion reduction. The additional saccharide-based moiety can be included to form metal nanoparticle suspensions that remain stably suspended for even longer periods (i.e., in comparison to suspensions stabilized with the capping agent alone) and can be a reducing sugar. The additional stabilizing agent is generally a monosaccharide, a disaccharide, or a derivative thereof. Suitable examples include sucrose, glucose, fructose, mannose, galactose, glyceraldehyde, lactose, and maltose, although the additional stabilizing agent more generally can include any combination of the saccharide residues listed above for the oligomeric/carbohydrate capping agent.

As described above, the carbohydrate-capped metal nanoparticles further include a functional group that is complementary to and capable of non-covalent bonding (e.g., hydrogen bonding) to a functional group on the oligonucleotide probe. For example, the functional group for the metal nanoparticles can be a carboxylic group, such as when the corresponding functional group of the oligonucleotide probe is an amino group. The carboxylic group can be in one or more of an acid form, a salt form, and an anionic (or dissociated) form. The functional group can be (covalently) attached to the metal nanoparticles via a thiol (—SH) group, which is particularly suitable for gold nanoparticles, or other group for other metals. Different functional groups for (covalent) attachment to a particular metal or metal alloy nanoparticle are generally known in the art and can be suitably selected by the skilled artisan. There is generally a linking group between the thiol group (or other group for metal nanoparticle attachment) and the carboxylic or other functional group for the metal nanoparticles, for example having linking groups with 2-20, 3-16, 4-12, or 6-10 carbon atoms (e.g., in an alkyl or other chain linker). In such cases, the functional group for the metal nanoparticles can be considered a pendant functional group relative to an outer surface of the metal nanoparticles.

Other complementary functional groups for the carbohydrate-capped metal nanoparticles and oligonucleotide probe are possible. For example, the functional group of the oligonucleotide probe can be a carboxylic group, and the functional group of the metal nanoparticles can be an amino group. This is representative of an alternative embodiment switching the amino and carboxylic functional groups, with a carboxylic-functionalized probe (e.g., oligonucleotide probe with a terminal carboxylic group) and amino-functionalized carbohydrate-capped metal nanoparticle (e.g., gold nanoparticle functionalized with a thiolamine including a hydrocarbon linking group between the thiol group and amino group). In another embodiment, the functional group of the oligonucleotide probe can be either a N-hydroxysuccinimide (NHS) ester or a carboxylic group, and the functional group of the metal nanoparticles can be the other of the N-hydroxysuccinimide (NHS) ester and the carboxylic group (e.g., NHS ester/carboxylic groups or carboxylic/NHS ester groups as complementary groups for non-covalent bonding). In another embodiment, the functional group of the oligonucleotide probe can be either biotin or (strept) avidin, and the functional group of the metal nanoparticles can be the other of biotin and (strept) avidin (e.g., biotin/(strept) avidin groups or (strept) avidin/biotin groups as complementary groups for non-covalent bonding).

The functional group can be added to the metal nanoparticles after their formation and combination with the carbohydrate capping agent. For example, a thiolated carboxylic acid can be combined with carbohydrate-capped gold nanoparticles under mild conditions (e.g., incubation at approximately room temperature with a surfactant) to form a gold-thiol attachment at the gold nanoparticle surface along with a pendant carboxylic group. For instance and as generally illustrated in the examples, 11-mercaptoundecanoic acid (11-MUDA) can be used to functionalize and attach a pendant carboxylic group to gold nanoparticles, but longer or shorter linking groups and/or different attachment groups (e.g., other than thiol for different metal nanoparticles) can be used as desired.

Stabilized Metal Nanoparticle Compositions: The above process results in the formation of a metal nanoparticle composition. Once the reduction reaction has progressed (e.g., to completion, such as once substantially all precursor metal ion reactant has been consumed), the aqueous medium contains a plurality of reduced metal nanoparticles as a suspension stabilized in the aqueous medium with the carbohydrate capping agent. Accordingly, the disclosure also relates to the use of a stabilized metal nanoparticle suspension composition that includes water in a sufficient amount to provide an aqueous medium and stabilized metal nanoparticles stably suspended in the aqueous medium. The aqueous medium suspension can have the same neutral or alkaline pH as that used for metal ion reduction (e.g., ranging from 7 to 12), or it can be adjusted to a different pH value post-reduction (e.g., still generally in the neutral or alkaline range) for storage. The stabilized metal nanoparticles in the suspension individually can include a metal nanoparticle core (e.g., generally having a spherical or nearly spherical/spheroidal shape) and a carbohydrate capping agent present as a layer on an outer surface of the metal nanoparticle core in an amount sufficient to stabilize the metal nanoparticle suspension (i.e., the capping agent need not completely envelop the nanoparticle core, but it is present near the core surface in a sufficient amount to prevent/inhibit substantial settling or agglomeration of the nanoparticles). Similarly, stabilized metal nanoparticles in the suspension individually can include a core-shell nanoparticle and a carbohydrate capping agent present as a layer on an outer surface of the metal nanoparticle shell in an amount sufficient to stabilize the metal nanoparticle suspension. In various embodiments, the carbohydrate capping agent can form a complete or partial layer (e.g., a monolayer or a plurality of layers) that is adsorbed or otherwise bound to the metal nanoparticle surface such as by electrostatic interactions between the metal nanoparticle surface and hydroxyl groups of the carbohydrate capping agent present at the neutral or alkaline pH of the aqueous medium.

The population of the reduced metal nanoparticles as produced (e.g., in suspension as formed in the aqueous medium or otherwise) generally has a particle size ranging from 2 nm to 50 nm (e.g., a number-, weight-, or volume-average particle size). For example, the average size of the nanoparticle distribution can be at least 2, 5, 8, 10, 12, or nm and/or up to 8, 10, 12, 15, 20, 25, 30, 40, or 50 nm. In an embodiment, the distribution of metal nanoparticles also has a relatively narrow size distribution, for example a substantially normal size distribution with a standard deviation of 25% or less relative to the average particle size of the distribution (e.g., a monomodal distribution; having a σ/<x> for a normal distribution of not more than 25%, 20%, 15%, or 10% and/or at least 1%, 2%, 5%, 8% or 10%). Various size parameters of the metal nanoparticle distribution (e.g., average size, distribution width) can be selected/controlled by selecting one or more reduction reaction parameters. Examples of suitable reaction/operating conditions that can be selected to control nanoparticle size include capping agent concentration, metal ion concentration, reducing agent concentration, reaction temperature, reaction pH, length and/or size distribution of the oligomeric capping agent.

The capping agent-stabilized metal nanoparticles remain stably suspended in the aqueous medium for extended periods without (substantial) settling or agglomeration of the nanoparticles. For example, the suspension can remain stable for at least 90 days when stored at room temperature. In various embodiments, the suspension is stable or capable of remaining stable for periods of at least 90,120,180, 270, or 360 days and/or up to 270, 360, 720, or 1080 days and/or at storage temperatures generally between 20° C. and 25° C., in particular at a neutral or alkaline pH. The metal nanoparticles remain stably suspended in the aqueous medium in part based on the hydrophilic character of various functional groups the carbohydrate capping agent (e.g., hydroxyl groups, which can impart a water-soluble character to low-molecular weight capping agents).

The capping agent-stabilized metal nanoparticles are suitably non-functionalized with respect to functional groups or moieties that can specifically bind or non-specifically bind to a target analyte. For example, the functionalized, carbohydrate-capped metal nanoparticle can be free from biomolecules and specific binding pair members as described in more detail below. Alternatively or additionally, the functionalized, carbohydrate-capped metal nanoparticle can be free from non-specific binding pair members which non-specifically bind to the target DNA analyte. Examples of such non-specific binding pair members include chitosan or other glycan such as in chitosan- or glycan-functionalized magnetic nanoparticles that can be used in a pre-step for sample extraction and/or concentration. This reflects the structure of the metal nanoparticles before addition to the sample mixture and in situ formation of the nanoparticle-probe adduct therein. The metal nanoparticle need only include a carboxylic, amino, or other complementary group for non-covalent binding with the functionalized oligonucleotide probe, but need not include any functionalizing components for specific binding of the particular target DNA analyte or for non-specific binding to biological targets in general.

In embodiments, the capping agent-stabilized metal nanoparticles are suitably free from biomolecules or specific binding pair members which specifically bind to a target analyte (e.g., a protein, virus, bacteria, ssDNA, such a DNA of a target microorganism or complementary ssDNA). A specific binding pair member generally includes one of two different molecules, each having a region or area on its surface or in a cavity that specifically binds to (i.e., is complementary with) a particular spatial and polar organization of the other molecule. The binding pair members can be referenced as a ligand/receptor (or antiligand) pair. These binding pair members include members of an immunological pair such as antigen-antibody. Other specific binding pairs such as biotin-avidin (or derivatives thereof such as streptavidin or neutravidin), hormones-hormone receptors, IgG-protein A, polynucleotide pairs (e.g., DNA-DNA, DNA-RNA), DNA aptamers, biomimetic antibody-antigen (e.g., molecularly imprinted synthetic polymer having specific binding capability with the antigen), and whole cells are not immunological pairs, but can be considered as binding pair members within the context of the present disclosure. Such biomolecules or specific binding pair members are often attached to nanoparticles by one or more of physical adsorption (e.g., resulting from electrostatic metal-biomolecule interactions), direct binding (e.g., based on affinity interactions between the metal and a functional group of the biomolecule, such as between a thiolated biomolecule and gold), covalent attachment (e.g., between the biomolecule and a covalent linking intermediate that is bound to the metal nanoparticle, such as through thiolated carboxylic acids, EDAC-mediated attachment of biomolecules, biotin-streptavidin linking, and azide-linking or other “click” functionalization techniques).

### Glycan-Coated Magnetic Nanoparticles

A functionalized magnetic particle composition according to the disclosure includes a magnetic particle core and a binding pair member bound to the magnetic particle core. The magnetic particle core (e.g., an iron oxide or otherwise as described below) is generally a nano- or micro-scale particle having a roughly spherical shape. The binding pair member (e.g., a glycan or component or derivative thereof or otherwise as described below) is suitably a biomimetic, non-specific binding ligand and is generally capable of non-specific binding to one or more biological target analytes. The biomimetic binding pair member is bound to an external or outer surface of the magnetic particle core, although some binding pair member can additionally be interspersed throughout the body of the magnetic particle core such that the binding capability to the target analytes results from the binding pair members that are at or on the outer surface of the magnetic particle core. Additionally, the binding pair member can be interspersed within magnetic nanoparticle clusters (e.g., also at external and/or internal surfaces thereof).

The magnetic particle core according to the disclosure is not particularly limited and generally includes any nano- or micro-sized magnetic particles (e.g., about 1 nm to about 1000 nm or 2000 nm) that can be magnetized with an external magnetic/electrical field. More generally, the magnetic particle core can have a particle size ranging from 1 nm to 2000 nm (e.g., at least 1, 2, 10, 20, 50, 100, 200, 300, 500, 800, or 1000 nm and/or up to 200, 300, 400, 500, 600, 800, 1000, or 2000 nm), where the particle size ranges can represent a range for the average particle size (e.g., a number-, volume-, or weight-based average particle size) and/or the particle size ranges can represent the span of the distribution (e.g., such as for all or substantially all particles; such as between the 10% and 90% sizes of the cumulative size distribution). The magnetic particles more particularly include superparamagnetic particles, which particles can be easily magnetized with an external magnetic field (e.g., to facilitate separation or concentration of the particles from the bulk of a sample medium) and then redispersed immediately once the magnet is removed (e.g., in a new (concentrated) sample medium). Thus, the magnetic particles are generally separable from solution with a conventional magnet. Suitable magnetic particles mainly include nano-sized iron oxide particles, in particular Fe₃O₄ (magnetite) or γ-Fe₂O₃; (maghemite). Such magnetic particles can be prepared by superparamagnetic iron oxide by precipitation of ferric and ferrous salts in the presence of sodium hydroxide and subsequent washing with water.

More generally, the magnetic particle core and corresponding magnetic particles can include a ferrimagnetic or ferromagnetic material (e.g., iron-containing particles providing electrical conduction or resistance). Suitable magnetic particles include iron-containing magnetic metal oxides, for example those including iron either as Fe(II), Fe(III), or a mixture of Fe(II)/Fe(III) (e.g., iron in a +2 and/or +3 oxidation state). Non-limiting examples of such oxides include an iron (II, III) oxide (Fe₃O₄; magnetite), an iron (II) oxide (FeO), or an iron (III) oxide (γ-Fe₂O₃; maghemite). The magnetic nanoparticles can also be a mixed metal oxide of the type M1ₓM2₃₋ₓO₄, wherein M1 represents a divalent metal ion and M2 represents a trivalent metal ion. For example, the magnetic nanoparticles may be magnetic ferrites of the formula M1Fe₂O₄, wherein M1 represents a divalent ion selected from Mn, Co, Ni, Cu, Zn, or Ba, pure or in admixture with each other or in admixture with ferrous ions. Other metal oxides include aluminum oxide, chromium oxide, copper oxide, manganese oxide, lead oxide, tin oxide, titanium oxide, zinc oxide and zirconium oxide, and suitable metals include Fe, Cr, Ni or magnetic alloys.

The binding pair member suitably is a biomimetic, non-specific binding ligand and is generally capable of non-specific binding to one or more biological target analytes. The binding pair member can include one or more glycans or glycoconjugates (e.g., glycoproteins, glycopeptides, peptidoglycans, glycolipids, glyco-oligonucleotides, glycosides, lipopolysaccharides) as well as components or fragments thereof (e.g., including one or more saccharide, protein, peptide, nucleotide, and/or lipid components from the glycans or glycoconjugates). Glycans generally consist of O-glycosidic linkages of monosaccharides, and glycoconjugates generally include a carbohydrate or polysaccharide portion covalently bonded to one or more other saccharide, protein, peptide, nucleotide, and/or lipid portions. Glycans, glycoconjugates, and components or fragments thereof in a natural setting can be involved in cell-cell interactions and recognition, and their inclusion as a binding pair member in the functionalized magnetic particle can provide corresponding biomimetic, non-specific binding capability for one or more biological target analytes (e.g., cell components thereof) to the functionalized magnetic particle. More generally, the binding pair member can function as a biomimetic pattern recognition receptor to distinguish pathogen-associated molecular patterns (e.g., lipopolysaccharide, mannose, peptidoglycans, lipoproteins, etc.) and facilitate non-specific binding to one or more biological target analytes. The biomimetic binding pair member is generally not capable of specific binding to a specific target analyte and does not typically include antibodies or oligonucleotides specific to a target analyte, for example.

In some embodiments, the binding pair member includes at least one of a carbohydrate moiety, an amino derivative thereof (e.g., amino sugar moiety or other carbohydrate/saccharide including an amine group substitute for a hydroxyl group, including amide (such as acetyl) derivatives), a carboxyl moiety (e.g., carboxylic acid and/or carboxylate salt thereof), and an amino acid moiety. Such binding pair members can be components of or otherwise derived from one or more glycans or glycoconjugates, The binding pair members can include monomeric, oligomeric (such as 2-10 residues), or polymeric forms of the various moieties, such as monosaccharides, disaccharides, oligosaccharides, and polysaccharides whether or not substituted with an amino group, as well as single amino acids, oligopeptides, and polypeptides. Examples of suitable binding pair members in the form of a carbohydrate moiety or an amino derivative thereof (e.g., as glycan components or otherwise) include one or more of a N-acetylglucosamine moiety, a N-acetylgalactosamine moiety, a N-acetylneuraminic acid moiety, a glucose moiety, a galactose moiety, a fucose moiety, a mannose moiety, a rhamnose moiety, a glucuronic acid moiety, a galacturonic acid moiety, an arabinofuranose acid moiety, and a xylose moiety. Examples of suitable binding pair members in the form of an amino acid moiety include one or more moieties from alanine, glycine, isoleucine, leucine, proline, valine, phenylalanine, tryptophan, tyrosine, aspartic acid, glutamic acid, arginine, histidine, lysine, serine, threonine, cysteine, methionine, asparagine, and glutamine (e.g., any desired amino acid). Examples of suitable binding pair members in the form of a carboxyl moiety include a carboxylic acid moiety and a carboxylate salt moiety (e.g., a sodium, potassium, or other alkali metal salt), such as in the form of a (meth)acrylic polymer coated on the magnetic particle core.

In a particular embodiment, the binding pair member comprises a N-acetylglucosamine moiety, for example as derived from chitosan. Chitosan is a linear polysaccharide composed of acetylated (N-acetyl-D-glucosamine) and deacetylated (β-(1→4)-linked D-glucosamine) glucosamine units. The amino sugar N-acetyl-D-glucosamine (GlcNAc or NAG) plays structural roles at a cell surface, being a component of bacterial and other cell wall peptidoglycans, fungal cell wall chitin, extracellular matrix of animal cells, and cell signaling. Chitosan also contributes to biofilm formation and substrate/intercellular adhesion. Chitosan can be incorporated into the functionalized magnetic particle as a binding pair member in one or more forms, including its original biopolymer/polymeric form (e.g., including both acetylated NAG units and deacetylated units), cleaved oligomeric units from the original biopolymer (e.g., including one or both of acetylated NAG units and deacetylated units, such as with 2 to 10, 20, or 50 monomeric units in the oligomer), cleaved monomeric units from the original biopolymer (e.g., including both acetylated NAG units and deacetylated units separately), and pyrolysis by-products of chitosan (e.g., including carbon and/or carbon nitride (aminated carbon)). Incorporation of chitosan into the functionalized magnetic particle in several different forms can result from a reaction process (e.g., a thermosolvo reaction process) to form the functionalized magnetic particle composition. Such reaction processes can be performed at about 200° C. for the synthesis of magnetic particle cores in the presence of a binding pair member source (e.g., chitosan biopolymer) to form the functionalized magnetic particle composition. Chitosan begins to degrade (e.g., depolymerize and/or pyrolyze) at about 200° C., thus resulting in incorporation of one or more of the original chitosan and foregoing degradation products into the functionalized magnetic particle composition. In addition to being a component of or derived from chitosan, N-acetylglucosamine can be incorporated as a monomer (e.g., from any suitable commercial source) as the binding pair member.

In some embodiments, the functionalized magnetic particle can include two or more different binding pair members, for example from the same or different general classes above (e.g., carbohydrate moiety, amino derivative thereof, carboxyl moiety, amino acid moiety). For example, in an embodiment, the functionalized magnetic particle can include a first binding pair member with an amino carbohydrate moiety (e.g., an N-acetylglucosamine moiety or other amine or amide carbohydrate derivative) and a second binding pair member with at least one of a carbohydrate moiety, a carboxyl moiety, and an amino acid moiety. Multiple different binding pair members can provide binding capability to a broader selection of biological target analytes. The inclusion of a select binding pair member can further improve size control and dispersion stability of the functionalized magnetic particle composition. For example, an amino carbohydrate moiety (such as an N-acetylglucosamine moiety or otherwise) can induce small particle size (about 200 nm or 300 nm to 500 nm or 600 nm with a narrow distribution) and can provide some hydrophilic surface groups to provide a stable, easily re-dispersible dispersion in water. The small size, stable dispersion, and binding capability of the functionalized magnetic particle composition makes it very fast to hybridize, capture, and magnetically separate various target analytes from a sample matrix.

A wide variety of biological target analytes may be bound/extracted by the functionalized magnetic particle composition, for example is a separation and/or concentration pre-step prior to detecting the target DNA analyte using the functionalized oligonucleotide probe and the complementarily functionalized carbohydrate capped metal nanoparticles (e.g., aminated probe and carboxylic-functionalized metal nanoparticles). Suitable biological target analytes can include one or more bacteria (e.g., whole and fragment), one or more viruses (e.g., whole and fragment), and/or one or more proteins (e.g., enzymes or other proteins). In some embodiments, the binding pair member can be selective for different classes of target analytes, such being capable of non-specific binding to multiple bacteria, but not generally to viruses or proteins (e.g., alternatively able to bind viruses but not bacteria or proteins, able to bind proteins but not bacteria or viruses, etc.). In further embodiments, the binding pair member can be selective within a class of target analytes, such as capable of non-specific binding to multiple types of bacteria, but not other types of bacteria (e.g., and likewise for virus and/or protein targets). Examples of specific biological target analytes of interest include one or more of Mycobacterium tuberculosis, Mycobacterium smegmatis (and other Mycobacterium species), Escherichia coli (various strains), Salmonella enteritidis (and other Salmonella species), Listeria monocytogenes, Vibrio cholera (and other Vibrio species) Bacillus cereus (and other Bacillus species), Dengue virus, influenza virus, and Newcastle disease virus. In some embodiments, the biological target analytes can include more generally Gram-positive and/or Gram-negative bacteria. In some embodiments, the functionalized magnetic particle composition can be used to extract fungi, plant diseases, and/or animal diseases from a plant or animal biological sample.

Suitably, the binding pair member is covalently bound to the magnetic particle core, for example including covalent attachment of the binding pair member to the magnetic particle material, such as an iron oxide component thereof. Typically, covalent binding can result from amino group-metal and/or hydroxyl group-metal covalent bonds, for example including —NH—Fe and —O—Fe bonds (e.g., resulting from reaction with —NH₂ and —OH groups present from chitosan with Fe present from an iron oxide magnetic particle core). In addition to chitosan, binding pair members more generally including NAG, carbohydrates, amino acids, etc. include amino groups and/or hydroxy groups, and they can similarly form covalent amino group-metal and/or hydroxyl group-metal bonds to the magnetic particle core. Adsorption of the binding pair member on the magnetic particle core is possible, but it is generally not the exclusive means for attachment. In some cases, covalent attachment is the exclusive means for attachment.

The particle size of the functionalized magnetic particle composition is not particularly limited. In some embodiments, the functionalized magnetic particle composition has a particle size ranging from 50 nm to 1000 nm. In other embodiments, the functionalized magnetic particle composition can have a particle size at least 50,100,200, 300, 500, 800, or 1000 nm and/or up to 200, 300, 400, 500, 600, 800, 1000, 2000, or 5000 nm. The particle size ranges can represent a range for the average particle size, such as a number-, volume-, or weight-based average particle size. Similarly, the particle size ranges can represent the span of the distribution, such as for all or substantially all particles, such as between the 10% and 90% sizes of the cumulative size distribution. Suitably, magnetic attraction between adjacent magnetic particle cores is weak enough such that van der Waals forces between the binding pair members are sufficient to prevent magnetic clumping or agglomeration of the corresponding functionalized magnetic particle composition when in the form of an aqueous dispersion. A corresponding nanoscale size of the functionalized magnetic particle composition (e.g., at least 100, 200, or 300 nm and/or up to 200, 300, 400, or 500 nm) is suitably selected to provide a colloidal suspension through Brownian motion.

The relative amounts of the magnetic particle core and the biomimetic binding pair member in the functionalized magnetic particle composition are not particularly limited. In some embodiments, a weight ratio of the magnetic particle core to the biomimetic binding pair member ranges from 4:1 to 1:4 (e.g., up to 10:1, 4:1, 2:1, 1.5:1 and/or up to 1:1.5, 1:2, 1:4, or 1:10). In other embodiments, the magnetic particle core and/or the biomimetic binding pair member independently can be present in the functionalized magnetic particle in amounts of at least 10, 20, 30, 40 or 50 wt. % and/or up to 50, 60, 70, 80 or 90 wt. % (e.g., about 30 or 40 wt. % to about 60 or 70 wt. %).

The specific form of the functionalized magnetic particle composition is not particularly limited. In some embodiments, the functionalized magnetic particle composition is in the form of a particulate powder, for example including a (dried) free-flowing powder that is generally free from liquids, such as synthesis solvents and/or end-use suspension liquids such as water. In other embodiments, the functionalized magnetic particle composition further includes water (e.g., distilled and/or deionized water) and is in the form of an aqueous suspension. The water provides a suspending medium for a plurality of functionalized magnetic particles in the form of a dispersion (e.g., a stable aqueous dispersion of the functionalized magnetic particles). The aqueous medium can include further components, such as buffer components for phosphate-buffered saline or otherwise. The functionalized magnetic particles suitably are present in the suspending medium at a concentration ranging from 0.01 g/L to 100 g/L (e.g., at least 0.01, 0.1, 1, 2, 5, or 10 g/L (or mg/mL) and/or up to 2, 5, 10, 20, 50, or 100 g/L (or mg/mL). Typical working concentrations that also form stable dispersions include 1 to 50 g/L, 2 to 20 g/L, 3 to 12 g/L, about 5 g/L, or about 10 g/L.

The functionalized magnetic particle composition includes a non-specific biomimetic binding pair member for one or more biological target analytes and need not necessarily include a specific binding pair member for one or more same or different biological target analytes. In some embodiments, the functionalized magnetic particle composition is free from specific binding pair members (e.g., specific binding pair members selective to one or more of the biological target analytes to which the non-specific biomimetic binding pair member can bind), such as antibody probes and/or oligonucleotide probes specific to a target analyte. In other embodiments, the functionalized magnetic particle composition can include a specific binding pair member for a desired target analyte. In such cases, the biomimetic binding pair member can serve an additional or alternative function, such as providing a means for particle size control and suspension stability, a reporter/label moiety, etc. In some embodiments, the functionalized magnetic particle composition is free from (pendant) amino groups, carboxylic groups, or other functional groups capable of non-covalent binding with the functionalized probe DNA and/or the functionalized, carbohydrate-capped metal nanoparticle.

The functionalized magnetic particle composition can be formed using any suitable method, for example including a one-pot solvothermal synthesis. Suitably, a magnetic particle precursor and a binding pair member precursor capable of non-specific binding to a plurality of biological target analytes are reacted in a non-aqueous reaction medium under sufficient temperature and pressure to form a functionalized magnetic particle composition according to the disclosure. The magnetic particle precursor can include a metal salt of a corresponding magnetic metal as generally described above, for example including at least one of an Fe(II) and an Fe(III) salt (e.g., an iron halide salt such as iron chloride, preferably in a hydrate form). The binding pair member precursor can include one or more of a glycan, a glyconjugate, and components thereof as generally described above. In various embodiments, the binding pair member precursor includes at least one of a carbohydrate, an amino derivative thereof, a carboxyl compound, and an amino acid compound as generally described above (e.g., for the corresponding moieties as incorporated into the functionalized magnetic particle). The reaction can be performed in any desired solvent, for example a polar organic solvent (e.g., ethylene glycol, other alcohols or diols), and can further include an initiator for metal (e.g., iron) oxide nucleation and magnetic particle core formation (e.g., sodium acetate).

The disclosed functionalized magnetic particle composition can be used to extract a biological target analyte from a sample. The functionalized magnetic particle composition is contacted with a sample containing or suspected of containing one or more biological target analytes to which the (biomimetic) binding pair member of the functionalized magnetic particle composition is capable of non-specific binding for a time sufficient to bind any biological target analytes in the sample to the functionalized magnetic particle composition. After a sufficient binding time, a magnetic particle-analyte conjugate is formed in the sample medium. In various embodiments, contacting the functionalized magnetic particle composition with the sample can be performed by adding the functionalized magnetic particle composition to the sample medium, adding the functionalized magnetic particle composition and the sample to a third aqueous or other fluid medium, etc. In various embodiments, the functionalized magnetic particle composition is contacted with the sample for a period ranging from 1 minute to 30 minutes before subsequent magnetic separation (e.g., contacting for at least 1, 2, 3, or 5 minutes and/or up to 5, 10, 15, 20, or 30 minutes to bind/extract any target analyte(s) present in the sample to the functionalize magnetic particles). Contacting or incubation for binding/extraction can be performed under mild conditions, such as room temperature (e.g., about 20-30° C.). The magnetic particle-analyte conjugate is then magnetically separated from the sample. For example, magnetic separation can include magnetically immobilizing the magnetic particle-analyte conjugate with an external magnet to the sample, removing/decanting sample supernatant, and then rinsing/washing the remaining sample with a suitable wash fluid (e.g., PBS) to remove any remaining sample matrix, and optionally re-suspending the magnetic particle-analyte conjugate in the wash fluid.

The sample to be tested and extracted is not particularly limited and can include any of a variety of biological materials, food products, environmental samples (e.g., environmental water or soil), surface swabs testing for contamination, etc. For example, the sample can be a biological material such as a sample of human or other animal tissue or fluid, for example including saliva, sputum, urine, blood, cerebrospinal fluid, tracheal swabs, etc. In other embodiments, the sample can be a food item, such as one or more of vegetables, fruits, eggs, poultry (chicken), fish, seafood, milk, mayonnaise, spinach, and components thereof. Food items suitably can be ground, shredded, otherwise reduced in size, etc. and/or added to a fluid sample medium to which the functionalized magnetic particle composition is also added to form the magnetic particle-analyte conjugate.

In various embodiments the method for extracting a biological target analyte from a sample can be extended to a method of detection as well, such as where the extraction method is used as a front end processing method for any desired (conventional) detection method to identify and/or quantify particular target analytes in the original sample. For example, the method can be extended by detecting a specific biological target analyte in the magnetic particle-analyte conjugate extract, such as by using any conventional analyte-specific probe or assay (e.g., with an analyte-specific antibody, oligonucleotide, etc.), which probe is optionally labeled with any suitable detection/reporter moiety, such as a visible reporter, an electrochemical reporter, an enzymatic reporter, etc. Alternatively or additionally, the method can be extended by detecting a non-specific biological target analyte in the magnetic particle-analyte conjugate. For example, visible observation of a matting/aggregation of the magnetic particle-analyte conjugate in the extracted sample can be is indicative of a non-specific bacterial infection in the sample (i.e., and correspondingly in a human patient, animal, or other organism from which the sample is obtained). Matting of the magnetic particle-analyte conjugate can be observed as a relatively large surface area sheet or thin aggregate of the conjugate, which forms or accumulates at or near the surface of a sample container in which the conjugate is formed or is present. Namely, the presence of such matting/aggregation of the magnetic particle-analyte conjugate need not generally identify the particular pathogen causing the infection, but nonetheless indicates the presence of some type of bacterial infection (e.g., as a non-specific positive/negative screening for infection without identification of the specific infection). Similarly, the absence of such matting/aggregation of the magnetic particle-analyte conjugate in the extracted sample is indicative of a lack of bacterial infection in the sample. Of course, if a non-specific biological target analyte is detected (e.g., by visible observation of matting/aggregation or otherwise), the magnetic particle-analyte conjugate extract can be further tested by one or more conventional methods to identify the specific biological target analyte as described above.

### Apparatus for Detecting a Target DNA Analyte

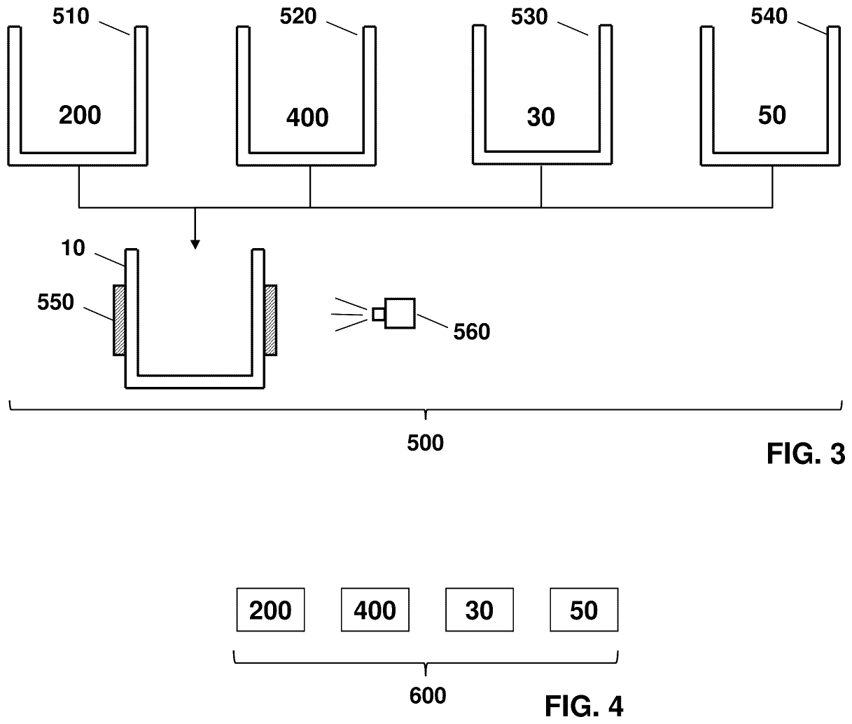

FIG. 3 illustrates an apparatus 500 for detecting a target DNA analyte 100 according to the disclosure. The apparatus 500 includes a sample chamber 10 adapted to receive the sample 20 to be analyzed for the presence or absence of the target DNA analyte 100, The sample chamber 10 can include a glass or other container with transparent wall(s) or wall section(s) for visual or (spectrophotometric) optical interrogation of the sample chamber 20. The apparatus 500 also includes at least one reservoir adapted to contain and deliver the aminated or other functional oligonucleotide probe 200 and the carboxylic-functionalized carbohydrate-capped metal nanoparticles 400 (e.g., aqueous or liquid suspension(s) thereof) to the sample chamber 20. As illustrated in FIG. 3, the probe 200 and the metal nanoparticles 400 can be stored in and delivered from separate reservoirs 510 and 520, respectively, such that the nanoparticle-probe adduct 300 is formed in the sample chamber 10. In another embodiment (not shown) the probe 200 and the metal nanoparticles 400 can be stored in and delivered from the same reservoir (e.g., already containing the nanoparticle-probe adduct 300 therein). The apparatus 500 can include one or more additional reservoirs, such as a reservoir 530 adapted to contain and deliver buffer 30 to the sample chamber 20 and a reservoir 540 adapted to contain and deliver acid 50 to the sample chamber 20. The apparatus 500 also can include a heating element 550 (e.g., an internal or external heating jacket or electrical heating elements) adapted to incubate the sample mixture 20 (e.g., denature and then anneal) in the sample chamber 10 after delivery of the sample 20, the functionalized oligonucleotide probe 200, and the functionalized carbohydrate-capped metal nanoparticles 400 (e.g., as an already-formed nanoparticle-probe adduct 300 or as separate components) to the sample chamber 10. The apparatus 500 also can include an optical interrogation means 560 adapted to colorimetrically detect metal nanoparticle destabilization and/or stabilization in the sample chamber 10 after delivery of the acid 50 to the incubated sample mixture 20. The optical interrogation means 560 can include a spectrophotometric detector such as including a light source of selected or controllable wavelength(s) and a light intensity detector. The optical interrogation means 560 also can include a (digital) camera to detect and analyze qualitative color changes in the sample chamber 10.

In an embodiment (not shown), the apparatus 500 can include a plurality of sample chambers 10 adapted to receive the various assay components More generally, the apparatus 500 can contain multiple sample chambers 10 to analyze multiple samples 20 for target analytes 100, either simultaneously or in (rapid) succession. The multiple samples 20 can be different samples (e.g., from different or distinct sources) to be analyzed for the same target analyte 100, in which case only one source/reservoir for a functionalized oligonucleotide probe specific to the target analyte 100 is needed. Alternatively or additionally, the multiple samples 20 can be aliquots or sub-samples of the same original sample (e.g., from the same source) to be analyzed for different target analytes 100, in which case multiple corresponding sources/reservoirs for corresponding functionalized oligonucleotide probes each specific to a particular target analyte can be included.

### Kit for Detection of a Target DNA Analyte

FIG. 4 illustrates a kit 600 for detection of a target DNA analyte 100 according to the disclosure. The kit 600 can include an aminated or other functionalized oligonucleotide probe 200 that is complementary to a target DNA analyte 100 and carboxylic- or other-functionalized, carbohydrate-capped metal nanoparticles 400. The kit 600 can further include a buffer 30 and/or an acid solution 50. The functionalized oligonucleotide probe 200 and the functionalized, carbohydrate-capped metal nanoparticles 400 can be present either in admixture or as separate components. The functionalized oligonucleotide probe 200 and the functionalized, carbohydrate-capped metal nanoparticles 400 can be present in relative amounts for forming a nanoparticle-probe adduct 300 upon combination (e.g., in selected/controlled relative nanoparticle: probe amounts or ratios (w/w or v/v), such as 1:2, 1:1.5, 1:1, 1.5:1, 2:1, or subranges thereof). As described above, the nanoparticle-probe adduct 300 is capable of binding the target DNA analyte 100. In some embodiments, the kit 600 can include multiple different functionalized oligonucleotide probes 200 (e.g., for detection of different target DNA analytes 100) for addition to/formation of a sample mixture 20 and corresponding nanoparticle-probe adduct 300. The different probes 200 can be combined with the metal nanoparticles 400 or with other probes in selected amounts or ratios as noted above.

### EXAMPLES

The examples illustrate the disclosed methods and compositions, but are not intended to limit the scope of any claims thereto. In particular, the examples illustrate methods of using aminated oligonucleotide probes in combination with carboxylic-functionalized carbohydrate-capped metal nanoparticles to detect a target analyte, in particular a target DNA analyte.

### Example 1—Synthetic and Analytical Methods

This example provides illustrative materials and methods for synthesizing various assay components and their use in detecting a target DNA analyte.

Glycan-coated MNP Synthesis: Glycan-coated magnetic nanoparticles (Fe₃O₄) (MNP) were synthesized. Glycan can be in the form of chitosan, mannose, glycine or other forms of amino acids or carbohydrates. The glycan-coated MNPs can be used pre-analysis extraction and/or concentration procedure to non-specifically bind, capture, and concentrate target DNA, which can then be detected using the aminated oligonucleotide probes and carboxylic-functionalized carbohydrate-capped metal nanoparticles according to the disclosure. The glycan-coated MNPs can be formed as follows: (1) In a 150-mL beaker, dissolve 2.0 g of ferric chloride hexahydrate (FeCl₃·6H₂O) in 75 mL of ethylene glycol with magnetic stirring until a clear solution is attained, about 10 min. Magnetic stirring is done by adding a small magnetic bar inside the beaker and placing the beaker on a stirring plate set at 350 rpm. (2) Then add 12.0 g of anhydrous sodium acetate, 0.8 g of chitosan. Stir the mixture vigorously (1,000 rpm on a stirring plate) for about 1 h at room temperature (about 20-30° C. or about 25° C.) until a homogeneous brown solution is observed. (3) Quickly pour the brown solution into a 200-mL TEFLON (or PTFE)-lined cup. (4) Insert the cup into a pressure/acid digestion vessel and cover the vessel. (5) Place the pressure vessel inside an oven and bake at 200° C. for 8 h. (6) After the reaction, switch off the oven and let the vessel completely cool to room temperature (overnight or about 8 h). (7) Using a 25-ml pipette attached to an electric pippetor, remove about 50 ml of the solvent (above the solution) and discard as waste. Then transfer the remaining solution to a 50-mL tube. (8) Wash the chitosan-coated Fe₃O₄ nanoparticles initially with 20-50 ml distilled water, and then sequentially with 20 ml distilled water followed by 20 mL of pure ethanol at least four times. (9) Place the 50-mL tube (uncovered) containing the solution in a vacuum oven and dry at about 40-60° C. for 12 h. (10) After drying, scrape dried nanoparticles and store in a clean dark glass vial.

Gold Nanoparticles (AuNP) Synthesis: Gold nanoparticle synthesis can be performed with the following reagents: dextrin stock solution, gold chloride stock solution, and sodium carbonate solution. Functionalization of AuNPs to incorporate a carboxylic functional group onto dextrin-capped AuNPs can be performed with 11-mercaptoundecanoic acid (11-MUDA), borate buffer, and sodium dodecyl sulfate (SDS) solutions.

Dextrin stock solution [25 g/L]: Dissolve 5 g of dextrin in 200 mL of distilled type 1 water in a 250-mL beaker with continuous stirring on a hot plate (100° C.) for 10-15 min (or until completely dissolved). Transfer the solution to a 250-mL orange-capped Pyrex bottle. Autoclave the bottle and store at room temperature.

Gold chloride (HAuCl₄) stock solution [0.1 g/12.5 mL]: HAuCl₄ is extremely hygroscopic and easy to oxidize; therefore, use a plastic spoon or a plastic-coated brown spoon to handle the powder and close the container rapidly in order to avoid oxygen contact. Weight 0.1 g of HAuCl₄ on a weighing boat. Add 12.5 mL of distilled sterile water type 1 into a 15 mL orange-capped tube. Take 1 mL of water from the orange cap tube and dissolve the HAuCl₄ in the weighing boat with it. Transfer the 1 mL yellow solution into the 15 mL tube, bringing the volume back to 12.5 mL. Cover with aluminum foil to prevent oxidation and store in the refrigerator until use. The gold chloride solution is preferably used within 24 hr for subsequent AuNP synthesis.

Sodium carbonate (Na₂CO₃) solution [10% w/v]: Weigh 5 g of Na₂CO₃ and dissolve in 50 mL of distilled type 1 water in a 100-mL orange-capped Pyrex bottle. Autoclave the bottle and store at room temperature.

11-mercaptoundecanoic acid (11-MUDA) solution [25 uM]: Prepare a stock solution of 11-MUDA (25 uM) by dissolving 1.10 mg 11-MUDA in 200 mL methanol. Store at room temperature.

Borate buffer [0.1 M, pH 7.4]: Add boric acid (H₃BO₃; 0.2 M) to borax (sodium tetraborate (Na₂B₄O₇·120H₂O); 0.2 M) solution until desired pH is reached. Dilute to desired molarity with ddH₂O. Adjust pH using HCl or NaOH. Autoclave for 15 minutes. Store at room temperature.

Sodium dodecyl sulfate (SDS) solution [0.025 M]: Prepare stock solution of SDS (0.5 M) by dissolving 6.60 g of SDS powder in 50 mL type 1 water. To prepare 0.025 M SDS, mix 500 μL of the stock solution in 9.5 mL type 1 water. Autoclave for 15 minutes. Store at room temperature.

One-Pot Synthesis of MUDA-Functionalized Dextrin-Capped Gold Nanoparticle (GNP-MUDA): (1) In a 125-mL Erlenmeyer flask, add 24.5 mL of distilled sterile type 1 water. (2) Add 5 mL of HAuCl₄ stock solution and mix by swirling the flask. The color of the solution will be yellow. The HAuCl₄ concentration in the reaction should be 2 mM (for HAuCl₄·3H₂O=0.04 g/50 mL). (3) Add 0.5 mL of 10% Na₂CO₃ in a dropwise manner while swirling the flask. Continue swirling the flask to mix and let stand for 3 min. At this stage, the solution looks greenish gray. (4) Add 20 mL of dextrin and mix by swirling the flask. Continue mixing until very pale yellow. (5) Add a magnetic stirring bar into the 125-mL flask and wrap the flask in aluminum foil to keep the solution dark/shielded from light. (6) Place the flask onto a hotplate at a setting of 150° C. pre-heated for about 20 min. Heat with stirring for 1 hour. The solution should turn into a bright red wine color. (7) Shut off heat and continue stirring for another 5 min to cool. (8) Remove 4 mL from the 50-mL GNP just synthesized and transfer to another tube. (The removed aliquot can be used as comparative reference for non-functionalized, dextrin-capped AuNPs (i.e., without MUDA).) (9) Add 2 mL 0.025 M SDS to the dextrin-capped AuNPs. Incubate with shaking for 30 min. (10) Add 2 mL 25 μM 11-MUDA (final concentration 1 uM). Incubate with shaking for 30 min to form GNP-MUDA. (11) Transfer 10 mL to 15-mL tubes and centrifuge GNP-MUDA at 10,000 rcf at 15° C. for 15 min. (12) Remove supernatant but leave about 50 uL liquid to prevent aggregation of AuNP. (13) Re-suspend in half the original volume using 0.1 M borate buffer pH 7.4. (14) Mix by inverting tubes three times. (15) Store in refrigerator until ready to use. For a scaled-up process (100 mL), the foregoing process can be performed by doubling the reagent amounts: 49 mL water, 10 mL HAuCl₄, 1 mL Na₂CO₃, 40 mL dextrin.

Bacterial Lysis and Genomic DNA Extraction: (1) Combine 1 mL of bacterial cell culture with 100 μL of glycan-coated MNP, pipette mix, and let stand for 5 min (similar to bacterial extraction). (2) Pipette mix and magnetically separate; discard the supernatant (bacteria will be attached to the glycan-coated MNP). (3) Re-suspend in 100 μL of distilled water (or less). (4) Boil for 10 min (to extract DNA). (5) Let cool for 10 min, magnetically separate; keep supernatant (containing the DNA). (6) Measure DNA concentration in supernatant using a NANODROP spectrophotometer. (7) Alternative method: After boiling, add 100 μL of 2-propanol and let stand for 5 min. Magnetically separate; discard supernatant. Wash with 70% ethanol; magnetically separate; discard supernatant. Re-suspend in 35 μL STE buffer. Incubate for 10 min at 65° C. While hot, magnetically separate immediately; transfer supernatant to a PCR tube. Measure DNA concentration in supernatant using the NANODROP spectrophotometer.

Gold Nanoparticle (GNP, GNP-MUDA) Characterization Procedure: (1) Obtain the absorbance spectrum of the synthesized gold nanoparticles using distilled or deionized water as standard. An absorbance peak should be observed at 520 nm (wavelength). (2) Characterize the particle size using TEM images and a ZETASIZER particle size analyzer (Malvern).

Aminated Oligonucleotide (Reporter) Probe: A customized oligonucleotide probe DNA that (a) has a desired/selected complementary sequence for target DNA binding, and (b) is aminated at the 5′-end of the oligonucleotide sequence is commercially available from Integrated DNA Technologies (Coralville, IA).

One-Pot Genomic DNA Detection: The following system and procedure can be adapted to high throughput analysis. (1) Prepare samples of target DNA (T), non-target DNA (N) and water control (C) in PCR tubes (3 replicates each). The Target DNA (T) sample includes 10 μL of T DNA. A mixture of T+non-target DNA (N) includes 5 μL of T DNA+5 μL of N DNA (similar DNA concentration). The non-target DNA (N) sample includes 10 μL of N DNA. The control (C) sample includes 10 μL of Type I deionized water. (2) Add 5 μL of 25 μM target probe and 5 μL of GNP-MUDA to each sample tube prepared above. (3) Place tubes in thermocycler and run using the following conditions: denaturation at 95° C. for 5 min; annealing at 55° C. for 10 min; cool at 25° C. (4) Add 5-10 μL of 0.1 M HCl to each tube. (5) Detection—observed via color change—A red or reddish purple color is observed for samples with T DNA (e.g., T DNA only or T+N mixed DNA). A blue or bluish purple color is observed for samples without T DNA (e.g., N DNA only or no DNA (control)). (6) Detection—observed via spectrophotometer—The light absorption spectrum can be measured about 3-10 min after HCl addition using a spectrophotometer. A measured light absorption peak at about 520 nm indicates the presence of at least some T DNA (e.g., via stabilized AuNP remaining dispersed in solution). A measured light absorption peak at or above about 600 nm indicates the absence of T DNA (e.g., via destabilized AuNP aggregating or agglomerating in solution). The foregoing process can be used for an unknown sample to determine the presence, relative amount, and/or absence of T DNA in the sample.

Alternative Embodiments: The chitosan coating on MNP for the glycan-coated MNPs can be replaced with any one or a combination of amino acids, carbohydrates, or other glycans (e.g. mannose, glucose, cellulose, etc.). Synthesis of glycan-coated MNPs is not limited to a solvothermal process, but may also include chemical, precipitation method, etc. Detection is not limited to target DNA, but could include target RNA. Detection is not only limited to bacteria, but also viruses, fungi, plants, clinical samples, and other biological materials.

### Example 2—Detection of Unamplified Bacterial DNA Extracted from Food Samples

This example illustrates the use of aminated oligonucleotide probes in combination with carboxylic-functionalized carbohydrate-capped metal nanoparticles according to the disclosure for the detection of E. coli O157 as a model foodborne pathogen. Rapid detection of foodborne pathogens such as E. coli O157 is essential in reducing the prevalence of foodborne illness and subsequent complications. In this example, a gold nanoparticle (GNP) biosensor was designed for visual differentiation between target (E. coli O157:H7) and non-target DNA samples. Results of DNA extracted from pure cultures indicate high specificity and sensitivity to as little as 2.5 ng/μL E. coli O157 DNA. Further, the biosensor successfully identified DNA extracted from flour contaminated with E. coli O157, with no false positives for flour contaminated with non-target bacteria. After genomic extraction, this assay can be performed in as little as 30 min. In addition, food sample testing was successful at detecting approximately 103 CFU/mL of E. coli O157 magnetically extracted from flour after only a 4 h incubation step. These results demonstrate the ability of the disclosed method to provide low-cost and rapid foodborne pathogen detection.

Materials: Frozen bacterial stock cultures of Escherichia coli O157, Salmonella enterica serovar Enteritidis, Bacillus cereus, and Listeria monocytogenes EGD-e were obtained from laboratories at Michigan State University (MSU). Escherichia coli C-3000 (15597) was obtained from the American Type Culture Collection (ATCC). The POWERLYZER MICROBIAL KIT and AE buffer solution used for DNA extraction were purchased from Qiagen (Germantown, MD, USA). A NANODROP ONE spectrophotometer from ThermoFisher Scientific was used to quantify DNA concentrations and absorbance spectra data (Waltham, MA, USA). The device has a working spectral range of 190-850 nm and wavelength accuracy of ±1 nm.

Chitosan-functionalized magnetic nanoparticles (200 nm in diameter) were used as received from the Nano-Biosensors Lab, MSU. WHIRLPAK bags (92 oz. and 18 oz.) were purchased from VWR International (Radnor, PA, USA). FLEXIMAG Separators were purchased from Spherotech Inc (Lake Forest, IL, USA). Tryptic Soy Agar (TSA), Tryptic Soy Broth (TSB), Hydrochloric acid (ACS reagent, 37%), gold (III) chloride tri-hydrate (HAuCl₄), sodium carbonate (Na₂CO₃), 11-mercaptoundecanoic acid (MUDA, HS(CH₂)₁₀CO₂H), sodium dodecyl sulfate (SDS, C₁₂H₂₅NaO₄S), and dextrin from potato starch (C₆H₁₂O₆) were purchased from Sigma Aldrich (St. Louis, MO, USA). Phosphate Buffer Solution (PBS), pH 7.4, was prepared as directed by the supplier, Sigma Aldrich.

Probe Design and PCR Confirmation: The oligonucleotide probe was designed to specifically target E. coli O157, with a genome size of approximately 5.5 Mb. The probe had an oligonucleotide sequence specifically targeting the Shiga toxin Stx1 subunit A (StxA1). The probe was designed using NCBI BLAST (National Center for Biotechnology Information Basic Location Alignment Search Tool) and purchased with 5′-amination and a poly-A tail from Integrated DNA Technologies (Coralville, IA, USA). Targeting the same gene, E. coli O157 primers (Stx1F934 and Stx1R1042) recommended by the Bacteriological Analytical Manual (BAM) were also purchased from Integrated DNA Technologies. For confirmation of biosensor results, PCR was conducted on pure E. coli O157 DNA samples and samples extracted from flour using the Qiagen POWERLYZER kit. The PCR protocol and gel electrophoresis was adapted from existing protocols amplifying Stx genes.

GNP Synthesis and Surface Coating: Dextrin-coated gold nanoparticles were synthesized using 5 mL of 2 mM gold (III) chloride trihydrate (HAuCl₄), sterile water, 0.5 mL of 10% sodium carbonate (Na₂CO₃) solution, and 20 mL of dextrin. The GNPs were thiol-coated using 25 μM MUDA to provide pendant carboxylic functionality, and then resuspended in 500 μL borate buffer.

Bacterial Culture: Frozen stock cultures of each bacterial species were stored at −80° C. Master plates were created by streaking 10 μL of a stock culture on TSA and incubating at 37° C. for 24-48 h. These plates were stored at 4° C. for a maximum of six weeks before replacement. Fresh bacterial cultures (or overnight transfers) were created for each experiment by transferring a single colony from the master plate into 9 mL TSB. Transfers were incubated overnight before use.

Biosensor Design: For each sample, 5 μL DNA probe, 10 μL sample DNA, and 5 μL GNPs were combined in a single tube. Samples were then heated in the thermocycler for probe hybridization. Tubes were subjected to 5 min at 95° C. (denaturing) and 10 min at 55° C. (annealing) before cooling to room temperature. This heating cycle causes target DNA (if present) to hybridize to the probe. After the tubes cooled, HCl was added. Application of salts such as HCl typically causes GNP aggregation; however, target DNA bound to the GNP-probe prevents this. Thus, samples with non-target DNA aggregated and turned purple/blue, while samples with target DNA remained red. This was quantified with absorbance spectra; red samples retained a peak wavelength close to 520 nm, while purple/blue samples shifted to higher peak wavelengths. This process is described above and illustrated in FIG. 1 (process steps) and FIG. 2 (red absorbance spectrum with bound target DNA in panel A; blue absorbance spectrum without bound target DNA in panel B).

Parametric studies were performed for the amount of HCl added and the time between HCl addition and reading colorimetric results (5-15 min). The HCl amount and aggregation time were determined through quantitative and qualitative analysis. First, HCl volume was varied by adding 5 μL 0.1 M HCl at a time to negative control (water) and target (10 ng/μL E. coli O157 DNA) tubes at 1 min intervals until aggregation of the control without aggregation of the target tube was visually observable. The lowest HCl volume with visible control tube aggregation was then used to compare target samples to multiple non-targets, all at 10 ng/μL. Absorbance spectra readings were taken at 5 min intervals after HCl application until visible aggregation of the target samples occurred. Thus, the final procedure had the greatest and most consistent peak shift difference between target and non-target samples, along with a visibly red target sample when compared to the non-target and control.

Sensitivity Testing: A series of 9 trials using a DNA concentration of 10 ng/μL was conducted to determine biosensor specificity. Four non-target bacterial species were represented: Escherichia coli C-3000, S. Enteritidis, Listeria monocytogenes, and Bacillus cereus. A negative control containing water and no DNA was also included. Genomic DNA extraction was achieved using the Qiagen POWERLYZER DNA extraction kit on overnight bacterial transfers. Extracted DNA was measured using NANODROP dsDNA measurements and diluted in AE buffer to 10 ng/μL. Ten minutes after HCl application, absorbance measurements and images were collected. Results were analyzed through quantification of “peak wavelength,” the wavelength corresponding to peak absorbance.

Specificity Testing: A separate series of 9 trials was conducted to determine biosensor sensitivity. DNA collected and quantified as previously described was serially diluted to lower concentrations, ranging from 20 to 1.25 ng/μL. For each replicate, a target DNA sample was compared to a non-target sample of the same concentration. The difference in peak wavelength between target and non-target samples was then calculated at each DNA concentration level. Statistical analysis of this peak wavelength difference was then used to determine sensitivity, where a peak wavelength difference between non-target and target samples significantly greater than zero (α=0.05) indicated sensitivity at this DNA concentration.

Biosensing from Large Food Samples: Bacteria extracted from food using a magnetic nanoparticle (MNP)-based extraction procedure was also tested in the biosensor. The extraction procedure was adapted to follow BAM (Bacteriological Analytical Manual) protocols, beginning with artificial contamination. Extractions were conducted in triplicate for E. coli O157 (T), L. monocytogenes (NT1), and E. coli C-3000 (NT2). In addition, a set of trials was conducted without artificial inoculation (NT3).

To contaminate samples, 1 mL of an overnight transfer was first added to 9 mL of TSB and incubated for 4 h. Then, 25 g of flour was weighed into a 92 oz. WHIRLPAK bag. Next, 1 mL of the 4 h bacterial culture was serially diluted to approximately 105 CFU/mL and added to the sample. This culture was further diluted and plated on TSA to confirm the initial concentration. After artificial contamination of the flour samples, bacteria were allowed to acclimate for 1 h at room temperature before 225 mL of PBS was added to each sample. Bags were then placed in a stomacher for 2 min, and the liquified food matrix was separated into WHIRLPAK bags with 100 mL of liquified food each. Then, 1 mL of MNPs were added to the bag, mixed, and allowed to incubate at room temperature. After 5 min, the WHIRLPAK bag was attached to a magnetic rack for another 5 min before supernatant removal. The remaining sample was resuspended in 1 mL PBS.

For each concentrated sample, 500 μL was then transferred to 4.5 mL of TSB and incubated for 4 h. DNA extraction was then performed using the Qiagen POWERLYZER kit and quantified using the NANODROP. Samples with the same bacterial inoculation were pooled for testing. DNA extracted from target-inoculated flour was then compared to two nontarget-inoculated DNA samples, as well as DNA from uncontaminated flour. Pooled DNA samples extracted from flour were compared at initial extraction concentrations. If initial concentrations between samples differed by >5 ng/μL, samples were diluted to the lowest concentration in the sample set for standardization and tested again.

Statistical Analysis: Statistical analysis utilized 95% confidence intervals of the wavelength corresponding to peak absorbance to compare target and non-target results. In addition, comparison of multiple groups for specificity and food testing was accomplished through the Kruskal-Wallis test and the non-parametric Student-Newman-Keuls test. Sensitivity testing relied on 95% confidence intervals using the Student's t distribution. Groups were tested with 9 replicates per sample (n=9) for all pure cultures and 6 replicates per sample (n=6) for studies in samples extracted from flour.

Colorimetric Biosensor Detection: The biosensor is based on the SPR band produced by the coherent oscillation of free electrons in colloidal GNP solutions. Upon aggregation of these GNPs, the distance-dependent nature of the SPR band leads to a color change from red to blue. The dextrin-coated GNPs utilized in this example displayed a clear absorbance peak at 520 nm, with shifts to higher peak wavelengths after HCl application. Application of HCl to the solution disrupted the electrostatic repulsion maintaining the colloid GNP suspension. This biosensor utilizes E. coli O157 DNA as the target analyte, which anneals to the probe-functionalized GNPs under thermocycler conditions described above. Even upon acid addition, DNA-GNP conjugates are protected from aggregation, leading to the output signal of a red solution in target samples. Non-target samples would turn purple/blue due to the lack of GNP protection by target DNA. Therefore, this biosensor can produce a quantifiable produce a quantifiable signal corresponding to the presence or absence of the target analyte. The signal can be measured visually or using a spectrophotometer.

Biosensor Specificity Results: As a result of the parametric study described above, 10 μL aliquots of 0.1 M HCl addition was used, and the analysis time was 10 min after HCl addition. Thus, all analysis steps from sample preparation to colorimetric analysis could be completed in approximately 30 min. This short duration is primarily due to the surface functionalization of the GNPs. As the coated GNPs have carboxylic acid (—COOH) groups, they form non-covalent interactions with the amine-functionalized DNA probes, leading to almost instantaneous GNP-probe functionalization.

Specificity of the biosensor was analyzed in a trial that included one water control, one target sample (E. coli O157), and four non-target species and strains: E. coli C-3000 (NT1), S. Enteritidis (NT2), L. monocytogenes (NT3), and B. cereus (NT4). All DNA samples were tested at a concentration of 10 ng/μL. The sample with target DNA (E. coli O157) had a reddish purple color after acid addition, exhibiting a shift increase in peak absorbance by about 64 nm (i.e., about 585 nm peak absorbance for samples with target DNA complexed with the GNPs vs. about 520 nm peak absorbance for original GNPs in suspension). The samples with non-target DNA (NT1-NT4) had a bluish color after acid addition, exhibiting a shift increase in peak absorbance by about 101-142 nm. Notably, the negative (non-detect) result for E. coli C-3000 (NT1) also demonstrated biosensor specificity for target strains within the E. coli species. As E. coli C-3000 does not contain the target gene (Stx1) or complementary sequence to the probe, the samples with E. coli C-3000 DNA display GNP aggregation consistent with a negative result. Accordingly, the biosensor can specifically detect Shiga-toxin-producing E. coli strains, which is useful because non-STEC E. coli strains that do not cause disease are often found in natural microflora.

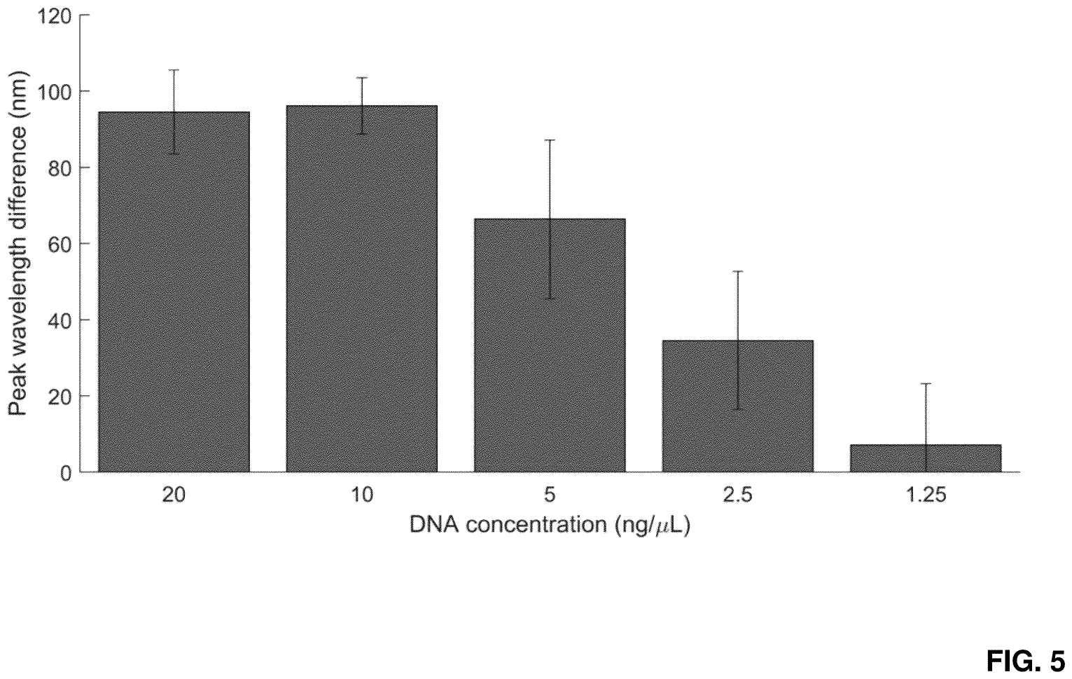

Biosensor Sensitivity Results: To determine the biosensor detection limit for E. coli O157, a sensitivity analysis was performed using non-target DNA (in this case, Listeria spp.) for comparison. Listeria monocytogenes is another dangerous foodborne pathogen. Both target and non-target DNA were diluted by a factor of two, with testing conducted between 20 and 1.25 ng/μL. Target and non-target samples of the same concentration were compared by their mean peak wavelength shifts. Resulting average differences between target and non-target peak wavelengths at each DNA concentration are graphically represented in FIG. 5 with 95% confidence intervals.

The results showed a statistically significant difference between target and non-target tubes at concentrations as low as 2.5 ng/μL. Although the lowest tested DNA concentration, 1.25 ng/μL, had a positive mean difference, the confidence interval overlapped zero. Therefore, detection at this concentration is not reliable. This indicates that the biosensor can reliably detect E. coli O157 target DNA concentrations at or above 2.5 ng/μL when compared to non-target samples of the same concentration.

Biosensor Detection from Flour Sample: Approximately 103 CFU/mL bacteria was extracted from artificially inoculated flour samples using glycan-coated MNPs. After 4 h of growth, DNA was extracted from the sample for implementation in the biosensor. In addition to E. coli O157-inoculated flour, DNA was extracted from flour inoculated with other foodborne pathogens (E. coli C-3000, NT1, and L. monocytogenes, NT2). Thus, testing simulated a situation in which only one bacterial species or strain was present in the food or sample purification steps had been taken. These samples were tested alongside a water control and one DNA sample extracted from magnetically separated flour that had not been artificially contaminated (NT3). DNA extractions from flour not inoculated produced a DNA concentration of 55.6 ng/μL, while the E. coli O157-contaminated flour sample produced a concentration of 83.4 ng/μL. Thus, it was concluded that this difference (approximately 28 ng/μL) is an estimate of the true target E. coli O157 DNA concentration in the sample. Both target samples (T and T60) exhibited significantly smaller peak shifts than all non-target samples, indicating successful detection of E. coli O157 from flour with an estimated 28 and 20 ng/μL of target DNA, respectively. With 95% confidence, the Kruskal—PCR amplification confirmed the presence of the target Stx1 gene in both pure cultures and DNA samples extracted from flour inoculated with E. coli O157.

Although the lowest detection limit of 2.5 ng/μL was achieved in pure cultures, the biosensor had a higher limit of detection (estimated between 10-20 ng/μL of target DNA) when detecting DNA extracted from flour. It is possible that DNA extracted from the food itself interferes with detection. Food particles were clearly visible in most concentrated samples, and the high concentration of DNA from pure flour samples despite the low concentration of natural microflora indicates that some food DNA was most likely extracted. Food particulates such as carbohydrates and fats are also known to interfere with DNA-based detection assays. This presence of food particles could potentially be addressed through upstream process modifications; for instance, washing the concentrated sample in PBS and repeating magnetic extraction (e.g., with glycan-coated MNPs) could reduce the food particles present in the sample selected for DNA extraction.

### Example 3—Detection of Unamplified DNA from Carbapenem-Resistant Bacteria

This example illustrates the use of aminated oligonucleotide probes in combination with carboxylic-functionalized carbohydrate-capped metal nanoparticles according to the disclosure for the detection of carbapenem-resistant bacteria. Antimicrobial resistance (AMR) is a global public health issue, and the rise of carbapenem-resistant bacteria needs attention. This example presents a nanoparticle-based plasmonic biosensor for detecting the carbapenemase-producing bacteria, particularly the beta-lactam Klebsiella pneumoniae carbapenemase (blaKPC) gene. The biosensor used dextrin-coated gold nanoparticles (GNPs) and an oligonucleotide probe specific to blaKPC to detect the target DNA in the sample within 30 min. The GNP-based plasmonic biosensor was tested in 47 bacterial isolates: 14 KPC-producing target bacteria and 33 non-target bacteria. The stability of GNPs, confirmed by the maintenance of their red appearance, indicated the presence of target DNA due to probe-binding and GNP protection. The absence of target DNA was indicated by the agglomeration of GNPs, corresponding to a color change from red to blue or purple. The plasmonic detection was quantified with absorbance spectra measurements. The biosensor successfully detected and differentiated the target from non-target samples with a detection limit of 2.5 ng/μL, equivalent to about 103 CFU/mL. The diagnostic sensitivity and specificity were found to be 79% and 97%, respectively. The GNP plasmonic biosensor is simple, rapid, and cost-effective in detecting blaKPC-positive bacteria.

Materials: A total of 47 bacterial cultures were used in this study: 3 bacteria from the American Type Culture Collection (ATCC), 38 carbapenemase-producing (CP) bacteria isolates from the Michigan Department of Health and Human Services (MDHHS), and 6 bacteria from Nano-Biosensors Laboratory at Michigan State University (MSU). DNA extraction kits were purchased from Qiagen (Germantown, MD, USA). NANODROP ONE from ThermoFisher Scientific (Waltham, MA, USA) was used to quantify DNA samples and absorption spectra. Oligonucleotide probes were obtained from Integrated DNA Technologies (IDT; Coralville, Iowa). Tryptic Soy Agar (TSA) and Tryptic Soy Broth (TSB), Hydrochloric acid (HCl), gold (III) chloride (HAuCl₄), sodium carbonate (Na₂CO₃), 11-mercaptoundecanoic acid (MUDA, HS(CH₂)₁₀CO₂H), sodium dodecyl sulfate (SDS, C₁₂H₂₅NaO₄S), and dextrin from potato starch (C₆H₁₂O₆) were purchased from Sigma Aldrich (St. Louis, MO, USA).

Bacterial Cultures: Bacterial strains of E. coli C-3000 (15597), KPC-producing carbapenem-resistant E. coli (BAA-2340), and Klebsiella pneumoniae subsp. pneumoniae (13883) were obtained from ATCC. Frozen cultures of Salmonella enterica serovar Typhimurium, Salmonella enterica serovar Enteritidis, Klebsiella pneumoniae, and Enterobacter cloacae, and carbapenem-resistant Klebsiella pneumoniae were obtained from MSU. The CP bacteria isolates from MDHHS included 12 KPC-producing bacteria: E. coli (2), E. cloacae (1), K. pneumoniae (3), K. aerogenes (2), Raultella ornithinolytica (2), Citrobacter amalonaticus (1), Citrobacter freundii (1), and 26 non-KPC (IMP, NDM, OXA-48, VIM)-producing bacteria: E. coli (5), K. pneumoniae (4), E. cloacae (7), K. oxytoca (1), C. freundii (2), Providencia rettgeri (2), Proteus mirabilis (2), Morganella morganii (2), and P. aeruginosa (1). Carbapenem-resistant bacteria isolates from MDHHS were verified by molecular (CARBA-R Cepheid assay and CDC laboratory-developed assay) and growth-based AST methods.

Stock cultures of all isolates were stored at −80° C. The cultures were refreshed by plating on TSA and incubated at 37° C. for 24-48 h. A single colony of the fresh bacterial cultures on TSA was then transferred into 9 mL of TSB with an overnight incubation at 37° C. before the experiment. The susceptible profile of E. coli C-3000, S. Typhimurium, S. Enteritidis, E. cloacae, K. pneumoniae, and K. aerogenes were confirmed using agar-dilution test (AST).

DNA Extraction: The DNA of the pure bacteria cultures after overnight incubation was extracted using the commercial kit, which removes any interfering materials and was finally suspended in elution buffer (pH 8). The DNA concentration and quality were measured with the NANODROP. DNA samples with acceptable A260/A280 and A260/A230 ratios, between 1.8 and 2.2, were used for the designed biosensor assay.

Probe Design and PCR Confirmation: A single-stranded oligonucleotide primer and probe were designed to target specifically KPC-producing bacteria. The primers and probe were designed using the blaKPC gene sequence of carbapenem-resistant E. coli (ATCC-2340), utilizing the design tools from NCBI BLAST (National Center for Biotechnology Information Basic Location Alignment Search Tool) and purchased with 5′-amination from Integrated DNA Technologies (Coralville, IA, USA). The absence and presence of the blaKPC gene in all samples were con-firmed by PCR; amplified products were run on a 2% agarose gel in Tris Acetate EDTA (TAE) buffer at an applied voltage of 120 V for 1 h.

GNP Synthesis and Surface Modification: Dextrin-coated gold nanoparticles (GNPs) were synthesized using an alkaline synthesis method. Briefly, gold (III) chloride trihydrate was dissolved in water and neutralized with sodium carbonate. Then, dextrin was added and heated at 150° C. under continuous stirring conditions until the solution turned wine red. The synthesis of GNPs was then confirmed by determining their absorption maxima using the NANODROP at around 520 nm (red color). The GNPs were modified with 25 μM mercaptoundecanoic acid (MUDA) and suspended in 0.1 M borate buffer. As the MUDA-coated GNPs have-COOH groups, they create non-covalent interactions with amine groups on the aminated probe, leading to almost instantaneous GNP-probe functionalization. Batches of the surface-modified, ready-to-use GNPs were stored at 4° C. until further use. Since the GNPs are stable for a long time, new synthesis is not required for everyday analysis.

Biosensor Design: The GNP-based plasmonic biosensor assay was developed with the following procedure [44]. Each biosensor trial included the extracted DNA sample (10 μL), 25 μM DNA probe (5 μL), and surface-modified GNPs (5 μL) in a single tube. Samples were then placed in a thermocycler to allow denaturation at 95° C. for 5 min, annealing at 55° C. for 10 min, and cooling to room temperature. This cycle enables target DNA to hybridize with the probe-GNP. Next, 0.1 M HCl was added to the sample, inducing GNP aggregation by distributing the electrostatic repulsion from the GNPs. However, target DNA bound to the GNP-probe prevents GNPs from aggregation. Thus, samples with target DNA remained red, while non-target samples allowed GNP aggregation, resulting in color change (purple or blue). The visual change in color of the GNPs was quantified by measuring their absorbance spectra in the wavelength range of 400-800 nm. Target samples were expected to have maximum absorbance at ˜520 nm, while blue/purple samples shifted right, with higher absorption maxima. Quantification of the GNP aggregation was determined using absorbance ratios at 625 nm and 520 nm (A625/520), which is based on an earlier reported study [50].

The GNP biosensor optimization variables included the amount of HCl (5-10 μL) and the response time between HCl addition and reading the colorimetric results (5-10 min). The optimum HCl amount and aggregation time were determined through qualitative and quantitative analysis. Different amounts of 0.1 M HCl (5-10 μL) were separately added to the negative control (nuclease-free water), positive sample (10 ng/μL of KPC-producing E. coli BAA-2340), and negative sample (10 ng/μL of E. coli C-3000). Tubes were incubated until aggregation of negative samples without aggregation of the positive sample, which was visually observable. Absorbance spectra readings were taken at 5 min intervals after HCl addition. Readings were statistically analyzed at a 95% confidence interval; the optimized procedure had a significant and consistent difference between positive and negative samples, with a visible color change.

Limit of Detection: The analytical sensitivity test was conducted at different DNA concentrations to determine the minimum detectable concentration of DNA. In this test, target and non-target DNA samples were serially diluted to lower concentrations, ranging from 20 to 1 ng/μL. Then, the target DNA sample (KPC-producing E. coli (BAA-2340)) was compared with a non-target sample (E. coli C-3000) at the same concentrations with a series of nine trials. Their visual color change and absorbance spectra measurements were used to determine the difference in GNP aggregations between the two samples. The A625/520 values were statistically analyzed at a 95% confidence interval.

Sensitivity results indicate that the plasmonic biosensor can detect and differentiate target and non-target samples at and above 2.5 ng/μL. The detection limit of the biosensor corresponds to approximately 103 CFU/mL, which is at least an order of magnitude more sensitive than representative lateral-flow immunochromatographic assays and electrochemical biosensors for CP bacteria detection, thus providing a more rapid and more sensitive assay for CP bacteria.

Sensitivity and Specificity Testing: The biosensor was tested with a total of 47 DNA samples: 14 KPC-producing bacteria (target DNA) and 33 non-KPC-producing bacteria (non-target DNA), as listed in Table 1. A DNA concentration of 10 ng/μL was used for all samples with a series of nine trials. Each specificity trial included a negative control (DNA-free), target, and non-target samples. Their absorbance spectra measurements and images were collected during the experiment. Differences in A_{625/520} values among target and non-target samples were statistically analyzed at a 95% confidence interval. The sensitivity is the proportion of positive tests (True positive/(True positive+False negative)), and specificity is the proportion of negative tests (True negative/(True negative+False positive)).

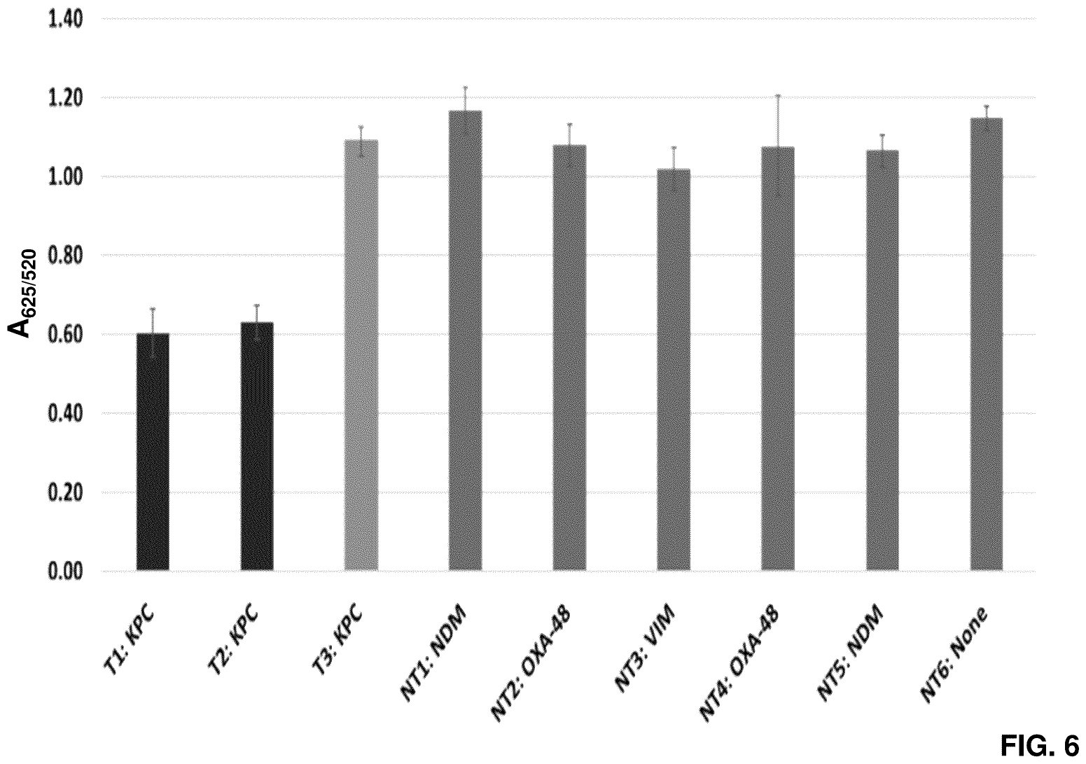

The tested 47 isolates were first confirmed for the presence of the blaKPC gene in the target (14) and its absence in non-target samples (33) by PCR amplification. The plasmonic biosensor was then tested on all target and non-target samples. FIG. 6 shows representative results from E. coli samples with three KPC-producing targets and six non-KPC-producing non-target samples. In the illustrative results of FIG. 6, the plasmonic biosensor successfully detected two of three target samples, and it successfully indicated the absence of the target analyte in six of six non-target samples. Overall, the plasmonic biosensor used the blaKPC probe to successfully detect and differentiate KPC-producing bacteria from non-KPC-producing bacteria regardless of bacterium types. For the total 47 tested isolates (not all results shown), the biosensor was in almost perfect agreement with the result of PCR amplification: The biosensor detected 11 of 14 target DNA samples and one of 33 non-target DNA samples, confirmed statistically (i.e., 11/14 correct positive determinations and 1/33 false positive determination). The average A_{625/520} of the detected target DNA samples in all trials was in the range of 0.43-0.63, while the absorbance ratio of non-target DNA detection was between 0.71-1.2 after HCl application (5 min response). The differences in absorbance ratio of all detected samples were significantly (p<0.05) different than those of all non-detected samples.

The diagnostic specificity (true negative) and sensitivity (true positive) of the biosensor were found to be 97% and 79%, respectively (i.e., 32/33 correct negative determinations and 11/14 correct positive determinations. These results were in the range of sensitivity and specificity levels of the phenotypic techniques used in clinical labs to detect carbapenem-resistant bacteria. For instance, MALDI-TOF MS detected the resistant bacteria with a range of 72.5-100% sensitivity and 98-100% specificity.

Principle of the GNP-Based Plasmonic Biosensor: The plasmonic biosensor concept is based on the SPR of GNPs, which can be determined spectrophotometrically. GNP aggregation results from the distribution of the electrostatic repulsion, leading to a shift in their absorption maxima due to the distance-dependent nature of the SPR. The shift in absorption maxima is associated with the color change from red to blue or violet. Thus, this example utilized GNPs' absorbance spectra at 520 nm and shift in the peak of maximum absorbance following HCl addition for DNA detection. It was hypothesized that DNA-probe-GNPs' conjugation would protect GNP against aggregation, leading to the maintenance of red color in the target DNA sample. In non-target samples, GNP would agglomerate due to a lack of protection by target DNA, changing their color from red to blue or purple. The biosensor thus produced a qualitative and quantitative signal. The quantifiable signal corresponds to the presence or absence of the target analyte. The plasmonic biosensor using the blaKPC probe was conducted with 9 μL of 0.1 M HCl and a 5 min response time for further analysis. The GNPs are prevented from aggregation for at least 30 min. All steps from sample preparation to colorimetric analysis can be completed in approximately 30 min; this short duration is due to instantaneous GNP-probe functionalization.

The biosensor using the blaKPC probe could be applied to more bacteria isolates to improve its sensitivity and specificity. The biosensor design could be extended to detect other carbapenemase genes using blaNDM, blaOXA-48, blaVIM, and blaIMP probes for broad-range detection. A multiplex GNP biosensor using multiple probes can be designed to detect all carbapenemase genes. Besides carbapenem resistance detection, the biosensor could be applied to other antibiotic-resistant bacteria (e.g., colistin, ampicillin, ESBL, etc.).

The colorimetric nature of the plasmonic biosensor offers rapid and simple visual detection in 30 min. The GNPs are easily prepared and modified and chemically stable for a long time. This assay has only three steps-adding extracted DNA to GNPs, placing the mixture in a thermocycler (acting as a heating block, not for DNA amplification), and HCl application. The estimated material cost of this GNP plasmonic biosensor is affordable, compared to rapid molecular methods and phenotypic methods. This GNP biosensor does not require PCR amplification, complex and costly equipment such as a spectrophotometer, mass spectrophotometer or qPCR, and data analysis. Other inexpensive phenotypic methods require overnight culture and are therefore not rapid. Hence, the biosensor platform is affordable, rapid, and broadly applicable, for example being useful for point-of-care testing and field studies.

Because other modifications and changes varied to fit particular operating requirements and environments will be apparent to those skilled in the art, the disclosure is not considered limited to the example chosen for purposes of illustration, and covers all changes and modifications which do not constitute departures from the true spirit and scope of this disclosure.

Accordingly, the foregoing description is given for clearness of understanding only, and no unnecessary limitations should be understood therefrom, as modifications within the scope of the disclosure may be apparent to those having ordinary skill in the art.

All patents, patent applications, government publications, government regulations, and literature references cited in this specification are hereby incorporated herein by reference in their entirety. In case of conflict, the present description, including definitions, will control.

Throughout the specification, where the compositions, processes, kits, or apparatus are described as including components, steps, or materials, it is contemplated that the compositions, processes, or apparatus can also comprise, consist essentially of, or consist of, any combination of the recited components or materials, unless described otherwise. Component concentrations can be expressed in terms of weight concentrations, unless specifically indicated otherwise. Combinations of components are contemplated to include homogeneous and/or heterogeneous mixtures, as would be understood by a person of ordinary skill in the art in view of the foregoing disclosure.

### PARTS LIST

10 sample or assay vessel
20 sample containing or suspected of containing a target DNA analyte
30 buffer solution
40 sample mixture
42 incubated solution
50 acid solution
60 solution-nanoparticle mixture
62 solution-nanoparticle mixture including stabilized metal nanoparticles (stabilized nanoparticle-probe-target DNA complexes)
64 solution-nanoparticle mixture including destabilized metal nanoparticles
100 target DNA analyte (or double-stranded DNA (dsDNA))
102 (denatured) first strand of dsDNA target analyte
104 (denatured) second strand of dsDNA target analyte
150 non-target DNA
152 (denatured) first strand of dsDNA non-target DNA
154 (denatured) second strand of dsDNA non-target DNA
200 aminated or other functionalized oligonucleotide probe (e.g., aminated probe DNA or single-stranded probe DNA (ssDNAp))
202 bound/hybridized oligonucleotide probe
204 free or unbound/non-hybridized oligonucleotide probe
300 nanoparticle-probe adduct
310 nanoparticle-probe-(target) DNA complex
400 carboxylic or other functionalized, carbohydrate-capped (stabilized) metal nanoparticles
410 stabilized metal nanoparticles
420 destabilized metal nanoparticles
500 apparatus for detecting a target DNA analyte
510 aminated oligonucleotide probe reservoir
520 carboxylic-functionalized carbohydrate-capped metal nanoparticle reservoir
530 buffer reservoir
540 acid reservoir
550 heating element
560 optical interrogation means (e.g., spectrophotometer, light, and/or (digital) camera)
600 kit for detection of a target DNA analyte

What is claimed is:

1. A method for detection of a target DNA analyte, the method comprising:
combining (i) a sample containing or suspected of containing a target DNA analyte with (ii) an aminated oligonucleotide probe that is complementary to the target DNA analyte and (iii) a carboxylic-functionalized carbohydrate-capped metal nanoparticle, thereby forming a sample mixture and a nanoparticle-probe adduct in the sample mixture;
incubating the sample mixture under conditions sufficient to bind the oligonucleotide probe with any target DNA analyte present in the sample mixture, thereby forming an incubated solution comprising (i) a nanoparticle-probe-DNA complex when the target DNA analyte is present in the sample, and (ii) the nanoparticle-probe adduct when the target DNA analyte is not present in the sample; and
combining the incubated solution with an acid in an amount sufficient to (i) destabilize any nanoparticle-probe adduct present in the incubated solution, and (ii) stabilize any nanoparticle-probe-DNA complex present in the incubated solution.

2. The method of claim 1, comprising:
detecting a relative degree of metal nanoparticle stabilization after combining the incubated solution with the acid.

3. The method of claim 2, wherein detecting a relative degree of nanoparticle stabilization comprises detecting a color state of the incubated solution after combination with the acid.

4. The method of claim 1, wherein the target DNA analyte comprises double-stranded genomic DNA (dsDNAg) characteristic of a target analyte organism.

5. The method of claim 4, wherein the target analyte organism is selected from the group consisting of a virus, a bacterium, a mould, a fungus, and a parasite.

6. The method of claim 1, wherein the sample comprises a cell containing DNA.

7. The method of claim 1, wherein the sample is selected from the group consisting of biological samples, environmental samples, industrial samples, and food samples.

8. The method of claim 1, wherein the sample comprises a biological sample, which is optionally selected from the group consisting of saliva, sputum, whole blood, serum, plasma, urine, tears, skin, cerebrospinal fluid (CSF), milk, cell cultures, viruses, bacteria, moulds, and fungi, parasites, tumors, plants, and food products.

9. The method of claim 1, comprising:
extracting a raw sample with a glycan-coated magnetic nanoparticle, thereby forming a sample concentrate; and
combining the sample concentrate, a portion thereof, or a fraction thereof with the aminated oligonucleotide probe and the carboxylic-functionalized carbohydrate-capped metal nanoparticle to form the sample mixture.

10. The method of claim 1, wherein the oligonucleotide probe is aminated at a 5′-end of the oligonucleotide probe.

11. The method of claim 1, wherein the oligonucleotide probe comprises a single-stranded oligonucleotide probe.

12. The method of claim 11, wherein the single-stranded oligonucleotide probe has a length of 5 to 100 nucleotide bases.

13. The method of claim 1, wherein the oligonucleotide probe (or ssDNAp) has a length in a range of 30 to 80 nucleotide bases.

14. The method of claim 1, wherein the sample mixture further comprises a buffer.

15. The method of claim 1, wherein the method does not include a DNA amplification step.

16. The method of claim 1, wherein the carboxylic-functionalized, carbohydrate-capped metal nanoparticle comprises a gold nanoparticle, a dextrin capping agent on an outer surface of the gold nanoparticle, and a thiolated carboxylic acid on an outer surface of the gold nanoparticle.

17. The method of claim 1, wherein the carboxylic-functionalized, carbohydrate-capped metal nanoparticle is free from (i) biomolecules and specific binding pair members which specifically bind to the target DNA analyte, and (ii) non-specific binding pair members which non-specifically bind to the target DNA analyte.

18. The method of claim 1, wherein the carboxylic-functionalized, carbohydrate-capped metal nanoparticle is in the form of a carboxylic-functionalized, stabilized metal nanoparticle suspension composition comprising:
water in sufficient amount to provide an aqueous medium; and
a plurality of stabilized metal nanoparticles stably suspended in the aqueous medium, each stabilized metal nanoparticle comprising: (i) a metal nanoparticle core, (ii) a carbohydrate capping agent present as a layer on an outer surface of the metal nanoparticle core in an amount sufficient to stabilize the metal nanoparticle suspension, and (iii) a carboxylic functional group on an outer surface of the metal nanoparticle core.

19. The method of claim 1, wherein incubating the sample mixture to form the incubated solution comprises:
denaturing the sample mixture under conditions sufficient to denature any target DNA analyte present in the sample mixture; and then
annealing the sample mixture under conditions sufficient to hybridize any denatured target DNA analyte present in the sample mixture with the oligonucleotide probe, thereby forming the nanoparticle-probe-DNA complex when the target DNA analyte is present in the sample.

20. The method of claim 1, combining the sample with the aminated oligonucleotide probe and the carboxylic-functionalized carbohydrate-capped metal nanoparticle comprises:
combining the aminated oligonucleotide probe and the carboxylic-functionalized carbohydrate-capped metal nanoparticle to form the nanoparticle-probe adduct; and then
combining the sample with the nanoparticle-probe adduct to form the sample mixture with the nanoparticle-probe adduct therein.

21. The method of claim 1, wherein destabilizing any nanoparticle-probe adduct present in the incubated solution comprises at least one of agglomerating and aggregating any nanoparticle-probe adduct present in the incubated solution.

22. The method of claim 1, wherein the acid comprises hydrochloric acid.

23. A stabilized complex suspension composition comprising:
water or buffer; and
a nanoparticle-probe-DNA complex stably suspended in the water or buffer:
wherein:
the nanoparticle-probe-DNA complex comprises: (i) a carboxylic-functionalized carbohydrate-capped metal nanoparticle, (ii) a target DNA analyte, and (iii) an aminated oligonucleotide probe that is complementary to the target DNA analyte;
the oligonucleotide probe is non-covalently bound to the carboxylic-functionalized carbohydrate-capped metal nanoparticle; and
the oligonucleotide probe is hybridized to the target DNA.

24. A kit for detection of a target DNA analyte, the kit comprising:
an aminated oligonucleotide probe that is complementary to a target DNA analyte;
a carboxylic-functionalized, carbohydrate-capped metal nanoparticle; and
optionally a buffer;
wherein:
the aminated oligonucleotide probe and the carboxylic-functionalized, carbohydrate-capped metal nanoparticle are present either in admixture or as separate components; and
the aminated oligonucleotide probe and the carboxylic-functionalized, carbohydrate-capped metal nanoparticle are present in relative amounts for forming a nanoparticle-probe adduct upon combination, the nanoparticle-probe adduct being capable of binding the target DNA analyte.

25. An apparatus for detecting a target DNA analyte, the apparatus comprising:
a sample chamber adapted to receive a sample to be analyzed for the presence or absence of a target DNA analyte;
at least one reservoir adapted to contain and deliver an aminated oligonucleotide probe and a carboxylic-functionalized carbohydrate-capped metal nanoparticle to the sample chamber, either in admixture or separately;
an additional reservoir adapted to contain and deliver acid to the sample chamber;
a heating element adapted to incubate the sample mixture in the sample chamber after delivery of the sample, the aminated oligonucleotide probe, and the carboxylic-functionalized carbohydrate-capped metal nanoparticle to the sample chamber;
optionally, an optical interrogation means adapted to colorimetrically detect metal nanoparticle destabilization and/or stabilization in the sample chamber after delivery of the acid to the incubated sample mixture.

26. The apparatus of claim 25, comprising a plurality of sample chambers, each sample chamber being adapted to (i) receive a separate sample to be analyzed for the presence or absence of the target DNA analyte, (ii) receive the aminated oligonucleotide probe and the carboxylic-functionalized carbohydrate-capped metal nanoparticle from the at least one reservoir, (iii) receive the acid from the additional reservoir, and (iv) be heated by the heating element to incubate the sample mixture therein.

27. A method for detection of a target DNA or RNA analyte, the method comprising:
combining (i) a sample containing or suspected of containing a target DNA/RNA analyte with (ii) a functionalized oligonucleotide probe that is complementary to the target DNA/RNA analyte and that comprises a first functional group, and (iii) a functionalized carbohydrate-capped metal nanoparticle comprising a second functional group complementary to and capable of non-covalent bonding to the first functional group, thereby forming a sample mixture and a nanoparticle-probe adduct in the sample mixture;
incubating the sample mixture under conditions sufficient to bind the oligonucleotide probe with any target DNA/RNA analyte present in the sample mixture, thereby forming an incubated solution comprising (i) a nanoparticle-probe-DNA/RNA complex when the target DNA/RNA analyte is present in the sample, and (ii) the nanoparticle-probe adduct when the target DNA/RNA analyte is not present in the sample; and
combining the incubated solution with a destabilizing agent in an amount sufficient to (i) destabilize any nanoparticle-probe adduct present in the incubated solution, and (ii) stabilize any nanoparticle-probe-DNA/RNA complex present in the incubated solution.

28. The method of claim 27, wherein:
the first functional group is an amino group; and
the second functional group is a carboxylic group.

29. The method of claim 27, wherein:
the first functional group is a carboxylic group; and
the second functional group is an amino group.

30. The method of claim 27, wherein:
the first functional group is either a N-hydroxysuccinimide (NHS) ester or a carboxylic group; and
the second functional group is the other of the N-hydroxysuccinimide (NHS) ester and the carboxylic group.

31. The method of claim 27, wherein:
the first functional group is either biotin or (strept) avidin; and
the second functional group is the other of biotin and (strept) avidin.

32. The method of claim 27, wherein the destabilizing agent comprises an acid.

33. A method for detection of a target DNA or RNA analyte, the method comprising:
combining (i) a sample containing or suspected of containing a target DNA/RNA analyte with (ii) a functionalized oligonucleotide probe that is complementary to the target DNA/RNA analyte and that comprises a first functional group, and (iii) a functionalized carbohydrate-capped metal nanoparticle comprising a second functional group complementary to and capable of non-covalent bonding to the first functional group, thereby forming a sample mixture and a nanoparticle-probe adduct in the sample mixture;
incubating the sample mixture under conditions sufficient to bind the oligonucleotide probe with any target DNA/RNA analyte present in the sample mixture, thereby forming an incubated solution comprising (i) a nanoparticle-probe-DNA/RNA complex when the target DNA/RNA analyte is present in the sample, and (ii) the nanoparticle-probe adduct when the target DNA/RNA analyte is not present in the sample; and
destabilizing any nanoparticle-probe adduct present in the incubated solution, while stabilizing any nanoparticle-probe-DNA/RNA complex present in the incubated solution.
